(19) United States
(12) Patent Application Publication
(10) Pub. No.: US 2012/0046363 A1
(43) Pub. Date: Feb. 23, 2012

(54) DOCOSAHEXAENOIC ACID FOR THE TREATMENT OF HEART FAILURE

(75) Inventor: William C. Stanley, Baltimore, MD (US)

(73) Assignee: University of Maryland, Baltimore, Baltimore, MD (US)

(21) Appl. No.: 13/318,597

(22) PCT Filed: May 10, 2010
(86) PCT No.: PCT/US2010/034212

§ 371 Date: Nov. 2, 2011

Related U.S. Application Data
(60) Provisional application No. 61/176,958, filed on May 11, 2009.

(51) Int. Cl. A61K 31/202; A61P 9/00; A61P 9/10

(52) U.S. Cl. 514/560

(57) ABSTRACT

There is currently no completely effective treatment for heart failure. Considering the need for, and current void in the medical field for, a treatment for heart failure, the invention is drawn to treating heart failure. In particular aspects, the invention is drawn to the discovery that certain polyunsaturated fatty acids (PUFAs) and doses thereof are useful for treating heart failure. In other particular aspects, the invention is drawn to the discovery that certain PUFAs and doses thereof are useful for preserving mitochondrial function in a heart failure subject.

### STATEMENT OF FEDERALLY SPONSORED RESEARCH AND DEVELOPMENT

This invention was made with government support under Grant No. R21 HL091307 and P01 HL074237 awarded by the National Institutes of Health. The government has certain rights in the invention.

### TECHNICAL FIELD

The invention relates to methods of treating heart failure. The invention further relates to compositions for treating heart failure.

### BACKGROUND OF INVENTION

Heart failure (HF) is defined by the American Heart Association as “a complex clinical syndrome that can result from any structural or functional cardiac disorder that impairs the ability of the ventricle to fill with or eject blood”³⁰. There are approximately 6 million patients in the US currently diagnosed with HF, and this number is growing with the aging of the population³⁰. Classically, HF patients have increased left ventricular (LV) mass, reduced cardiac contractility, and impaired LV filling (“diastolic dysfunction”)³¹. Most heart failure patients have a history of hypertension (˜80%), and many have LV hypertrophy³². Approximately 50-60% of heart failure patients have an enlarged LV end diastolic volume and low ejection fraction, while 40-50% have a normal ejection fraction and end diastolic volume^{33, 34}. Current medical therapies for HF are aimed at suppressing neurohormonal activation (e.g. angiotensin converting enzyme inhibitors, angiotensin II receptor antagonists, β-adrenergic receptor antagonists, and aldosterone receptor antagonists), and reducing fluid volume overload and symptoms (diuretics, digoxin, inotropic agents). These pharmacotherapies improve clinical symptoms and slow progression of contractile dysfunction and expansion of LV chamber volume, but nevertheless HF progression continues and prognosis for even optimally-treated patients remains poor³⁵⁻³⁷. Moreover, more intense suppression of the neurohormonal systems does not provide further benefit compared to more modest therapy³⁸⁻⁴⁰. Thus there is a need for novel therapies for HF that act independent of the neurohormonal axis that can improve cardiac performance and prevent or reverse the progression of LV dysfunction and remodeling⁴¹⁻⁴⁴. Nutritional approaches such as supplementation with omega-3 polyunsaturated fatty acids (PUFA) that act through mechanisms independent of current approaches are particularly attractive because they could work additively with current therapies while not exerting negative hemodynamic effects^{41-43, 45, 46}.

The most widely available omega-3 PUFA for human intake is α-linolenic acid (ALA), which is found in plant oils, specifically flaxseed oil (˜55% ALA), canola oil (˜11% ALA) and soy bean oil (˜7% ALA). The omega-3 PUFAs eicosapentanoic acid (EPA) and docosahexaenoic acid (DHA) are more rare, found primarily in oily fish, though DHA is also found in eggs, mother's milk, and algae. ALA can be converted to EPA in mammalian cells, however this conversion is low (˜10%)^{47, 48}. Epidemiological studies show that high intake of fish rich in EPA+DHA (greater than ˜1.5 g/day) is associated with a decrease in plasma triglycerides and less coronary heart disease⁴⁹⁻⁵¹. ALA shows a similar relationship with coronary heart disease, though a much higher level of consumption is required and the maximal effect is not as great^{47, 52-54}. EPA+DHA supplementation causes a dose dependent incorporation of EPA and DHA into membrane phospholipids in blood cells and myocardium in humans, with a plateau level for EPA and DHA incorporation in cardiac phospholipids reached by ˜25-30 days after initiation of oral supplementation^{55, 56}. Double blind placebo controlled clinical trials show that treatment with EPA+DHA from fish oil reduces serum triglyceride and fatty acid concentrations in a dose-dependent manner, and decreases sudden cardiac death, endothelial dysfunction and vascular inflammation^{49, 51, 57-69}. Supplementation with EPA+DHA also exerts anti-inflammatory effects in clinical studies, and has anti-aggregatory effects due to lowering of tissue phospholipid levels of arachidonic acid and thromboxane production^{28, 56, 70-72}. EPA+DHA is FDA approved for the treatment of hypertriglyceridemia at a dose of 3.4 g/d, and is widely used for this purpose in the US and Europe.

There is little information regarding the effects of omega-3 PUFA on cardiac contractile function, LV volume, myocardial energy metabolism or mitochondrial structure and function in HF, and there is virtually nothing known about the effects of supplementation of EPA and DHA when each is given alone. A large epidemiological study that followed older people for a 12-year period found a 31% reduction in the risk for developing HF in individuals consuming fish three to four times per week compared to those eating fish only once a month or less³. The reduction in the risk for HF was dependent on the estimated intake of EPA+DHA. Consumption of tuna or other broiled or baked fish was also associated with a lower heart rate, lower systemic vascular resistance, and greater stroke volume, and a higher E/A ratio reflecting superior diastolic function as assessed by echocardiography⁷³.

Recent results from the Gruppo Italiano per lo Studio della Sopravvivenza nell'Infarto miocardico (GISSI) HF trial showed favorable effects on morbidity and mortality with supplementation with a low dose of EPA+DHA in a large population of HF patients⁹. This randomized, double-blind, placebo-controlled trial was the first to investigate whether omega-3 PUFA could improve morbidity and mortality in a large population of symptomatic HF patients. Patients with New York Heart Association class II-IV HF (ejection fraction 33±8%, 63% NYHA Class II) were randomly assigned to fish oil (0.85 g/day of EPA+DHA) (n=3494) or placebo (n=3481) and followed up for a median of 3.9 years. The fish oil supplement used in this study is approved by the Food and Drug Administration for the treatment of hypertriglyceridemia, and contains EPA and DHA at a ratio of 45:55. Importantly, the dose used in the GISSI-HF trial was only 25% of the FDA approved dose for treating hypertriglyceridemia. Primary endpoints of the trial were all cause mortality, and time to death or admission to hospital for cardiovascular reasons. The treatment was well tolerated, and showed a significant reduction in mortality (adjusted hazard ratio 0.91, p=0.041) and admission to hospital for cardiovascular reasons (adjusted HR 0.92, p=0.009). The results demonstrate that long-term administration of a low dose of EPA+DHA reduced both all-cause mortality and admissions to hospital for cardiovascular reasons. While the benefit was modest, it is important to keep in mind that the dose was very low, and the study population was aggressively treated with β-adrenergic receptor antagonist (65%) and angiotensin converting enzyme inhibitors or angiotensin receptor antagonists (93%). In addition, a parallel GISSI-HF study with the rosuvastatin performed in a similar patient population found absolutely no beneficial effect^{74, 75}.

Current drugs for heart failure (HF) are near their limit and novel approaches are needed^{7, 8}. We showed that EPA+DHA dose-dependently prevented development of HF in response to chronic pressure overload, specifically left ventricular chamber expansion and dysfunction². The mechanism(s) for the beneficial effect is unclear, but could be due to improved mitochondrial function. Mitochondrial dysfunction contributes to cardiac pathology in HF through impaired transfer of chemical energy to contractile work and greater opening of the mitochondrial permeability transition pore (MPTP)^{10, 11}. MPTP promotes energy wasting and triggers cardiomyocyte death^{4, 12, 13}. Ex vivo hearts from rats fed EPA+DHA have improved LV mechanical efficiency, and our studies showed EPA+DHA suppressed MPTP formation and increased mitochondrial cardiolipin (CL)^{5, 14}. CL is an inner membrane tetra-acyl phospholipid that anchors cytochrome c to the membrane and prevents death¹⁵⁻¹⁹. Formation of respiratory supercomplexes (comprised of complex I, III & IV) are required for normal ETC flux and oxidative phosphorylation (ox phos), and are decreased in HF¹. CL is required for formation of supercomplexes, thus omega-3 PUFA may improve mitochondrial and LV function in HF by increasing CL and supercomplex assembly, preventing MPTP formation and cardiomyocyte death, and improving the transfer of chemical energy to contractile work²⁰⁻²³.

Most fish oil supplements contain both EPA and DHA. However, recent work shows that EPA and DHA have different effects on mitochondrial function, and metabolism²⁴⁻²⁹. Supplementation with a 30/70 mix of EPA/DHA increased DHA incorporation into CL 4-fold in normal rats, however little EPA was incorporated. Thus supplementation with solely DHA may be superior to EPA alone or the typical mix of EPA and DHA. Taken together, and considering the unmet need in the art for effective treatments for HF, the invention is drawn to the use of a composition comprising an omega-3 PUFA for the treatment of HF and mitochondrial dysfunction.

### BRIEF SUMMARY OF INVENTION

The invention relates to compositions comprising, consisting of, and consisting essentially of a PUFA. The invention further relates to administering a composition of the invention for treating heart failure.

In certain embodiments, the invention is drawn to administering a composition of the invention to a subject in need thereof for the treatment of heart failure. In particular embodiments, the composition comprises, consists of, or consists essentially of, an omega-3 PUFA. In other particular embodiments, the omega-3 PUFA is alpha-linolenic acid (ALA), eicosapentaenoic acid (EPA), docosahexaenoic acid (DHA), or any combination thereof. In further particular embodiments, the omega-3 PUFA is DHA.

In certain embodiments, the invention is drawn to preserving mitochondrial function. In particular embodiments, preserving mitochondrial function is achieved in a subject that has heart failure. In further embodiments, the subject that has heart failure has been diagnosed as having heart failure. In other particular embodiments, preserving mitochondria is a result of alterations in mitochondrial phospholipid composition and structure, specifically an increase DHA, EPA or cardiolipin (CL), or a change in CL composition.

In certain embodiments, the invention is drawn to a method of treating heart failure in a subject, comprising administering a pharmaceutical composition comprising a therapeutically effective amount of DHA to a subject in need of treatment. In related aspects, the pharmaceutical composition consists essentially of a therapeutically effective amount of DHA, or the pharmaceutical composition consists of a therapeutically effective amount of DHA. In this embodiment, the treatment contributes to one or more effects selected from the group consisting of preserving cardiac mitochondrial function, maintaining cardiac respiratory supercomplexes, increasing cardiac respiratory supercomplexes, maintaining cardiac oxidative phosphorylation, increasing cardiac oxidative phosphorylation, preventing cardiac MPTP opening, maintaining cardiac levels of DHA, increasing cardiac levels of DHA, maintaining levels of EPA, increasing levels of EPA, maintaining levels of cardiac mitochondrial cardiolipin, increasing levels of cardiac mitochondrial cardiolipin, inhibiting cardiomyocyte death by apoptosis and inhibiting cardiomyocyte death by necrosis. Alternatively, in this embodiment the treatment contributes to one or more clinical end points selected from the group consisting of reduced left ventricular volume, improved cardiac contractile function, reduced cardiac-related hospitalization, reduced cardiac-medical complications, improved quality of life and reduced mortality.

In a related embodiment, the invention is drawn to a method of treating heart failure in a subject, comprising administering a pharmaceutical composition comprising a therapeutically effective amount of DHA and a therapeutically effective amount of EPA to a subject in need of treatment. In related aspects, the pharmaceutical composition consists essentially of a therapeutically effective amount of DHA and a therapeutically effective amount of EPA, or the pharmaceutical composition consists of a therapeutically effective amount of DHA and a therapeutically effective amount of EPA. In this embodiment, the treatment contributes to one or more effects selected from the group consisting of preserving cardiac mitochondrial function, maintaining cardiac respiratory supercomplexes, increasing cardiac respiratory supercomplexes, maintaining cardiac oxidative phosphorylation, increasing cardiac oxidative phosphorylation, preventing cardiac MPTP opening, maintaining cardiac levels of DHA, increasing cardiac levels of DHA, maintaining levels of EPA, increasing levels of EPA, maintaining levels of cardiac mitochondrial cardiolipin, increasing levels of cardiac mitochondrial cardiolipin, inhibiting cardiomyocyte death by apoptosis and inhibiting cardiomyocyte death by necrosis. Alternatively, in this embodiment the treatment contributes to one or more clinical end points selected from the group consisting of reduced left ventricular volume, improved cardiac contractile function, reduced cardiac-related hospitalization, reduced cardiac-medical complications, improved quality of life and reduced mortality.

In certain embodiments, the invention is drawn to a method of increasing cardiac mitochondrial DHA, EPA or cardiolipin in a subject, comprising administering a pharmaceutical composition comprising a therapeutically effective amount of DHA to a subject in need thereof. In related aspects, the pharmaceutical composition consists essentially of a therapeutically effective amount of DHA, or the pharmaceutical composition consists of a therapeutically effective amount of DHA.

In a related embodiment, the invention is drawn to a method of increasing cardiac mitochondrial DHA, EPA or cardiolipin in a subject, comprising administering a pharmaceutical composition comprising a therapeutically effective amount of DHA and a therapeutically effective amount of EPA to a subject in need thereof. In related aspects, the pharmaceutical composition consists essentially of a therapeutically effective amount of DHA and a therapeutically effective amount of EPA, or the pharmaceutical composition consists of a therapeutically effective amount of DHA and a therapeutically effective amount of EPA.

In certain embodiments, the invention is drawn to a method of suppressing cardiac MPTP opening in a subject, comprising administering a pharmaceutical composition comprising a therapeutically effective amount of DHA to a subject in need thereof. In related aspects, the pharmaceutical composition consists essentially of a therapeutically effective amount of DHA, or the pharmaceutical composition consists of a therapeutically effective amount of DHA.

In a related embodiment, the invention is drawn to a method of suppressing cardiac MPTP opening in a subject, comprising administering a pharmaceutical composition comprising a therapeutically effective amount of DHA and a therapeutically effective amount of EPA to a subject in need thereof. In related aspects, the pharmaceutical composition consists essentially of a therapeutically effective amount of DHA and a therapeutically effective amount of EPA, or the pharmaceutical composition consists of a therapeutically effective amount of DHA and a therapeutically effective amount of EPA.

In certain embodiments, the invention is drawn to a method of treating cardiac mitochondrial dysfunction in a subject, comprising administering a pharmaceutical composition comprising a therapeutically effective amount of DHA to a subject in need thereof. In related aspects, the pharmaceutical composition consists essentially of a therapeutically effective amount of DHA, or the pharmaceutical composition consists of a therapeutically effective amount of DHA.

In a related embodiment, the invention is drawn to a method of treating cardiac mitochondrial dysfunction in a subject, comprising administering a pharmaceutical composition comprising a therapeutically effective amount of DHA and a therapeutically effective amount of EPA to a subject in need thereof. In related aspects, the pharmaceutical composition consists essentially of a therapeutically effective amount of DHA and a therapeutically effective amount of EPA, or the pharmaceutical composition consists of a therapeutically effective amount of DHA and a therapeutically effective amount of EPA.

In preferred aspects of each of the relevant embodiments on the invention, the therapeutically effective amount of DHA is between about 0.5 g and about 5 g, and the pharmaceutical composition is administered to the subject once or twice daily.

In preferred aspects of each of the embodiments on the invention, the pharmaceutical composition is administered to the subject as an oral formulation.

The foregoing has outlined rather broadly the features and technical advantages of the present invention in order that the detailed description of the invention that follows may be better understood. Additional features and advantages of the invention will be described herein, which form the subject of the claims of the invention. It should be appreciated by those skilled in the art that any conception and specific embodiment disclosed herein may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that any description, figure, example, etc. is provided for the purpose of illustration and description only and is by no means intended to define the limits the invention.

### BRIEF DESCRIPTION OF DRAWINGS

FIG. 1. Separation of supramolecular assemblies of mitochondrial oxidative phosphorylation complexes (“supercomplexes”) by one-dimensional BN-PAGE in interfibrillar heart mitochondria from a normal control and HF dog. LEFT-Complex V (C V). The density of the band corresponding to supercomplex CI-CIII2-CIV and complexes I, III, and IV were normalized to complex V band. *P<0.05 control vs. HF. #P<0.07 control vs. HF. From Rosca et al, Cardiovasc Res, 2008¹.

FIG. 2. Content of EPA and DHA in cardiac phospholipids as measured by gas chromatography. Rats fed the Standard Chow were either sham (open bars) or aortic banded (black fill). From Duda et al, Cardiovasc. Res. 2009².

FIG. 3. Cardiomyocyte apoptosis by TUNEL staining after 12 wks of aortic banding. #p<0.05 vs. sham standard chow, *p<0.05 vs. AAB standard chow. From Duda et al, Cardiovasc. Res. 2009².

FIG. 4. LV end diastolic and systolic volumes and the ratio of myosin heavy chain (MHC) β to MHCα at 11 wks in rats fed standard chow, or supplemented with EPA+DHA. *p<0.05 vs banded rats fed standard chow; #p<0.05 vs sham rats fed standard chow. From Duda et al, Cardiovasc. Res. 2009².

FIG. 5. Male Wistar rats were treated with a standard lab chow or supplemented with fish oil that was high in EPA and DHA (2.3% of energy intake as EPA+DHA). Differences were assessed with a 2-way ANOVA. SSM, subsarcolemmal mitochondria; IFM, intrafibrillar mitochondria. n=3 rats/group.

FIG. 6. The concentration of extra-mitochondrial Ca²⁺ in isolated cardiac subsarcolemmal mitochondria plotted as a function of the amount of Ca²⁺ infused into the cuvette. Bottom Panel: The percent of preparation with the MPTP, where MPTP was defined as the cumulative Ca²⁺ load when the concentration of extra-mitochondrial Ca²⁺ exceeded twice the steady state value. Note: Similar results were observed in isolated intrafibrillar mitochondria.

FIG. 7. Serum triglyceride (Left) and free fatty acid concentration (Right). Data are means of n=6-8/group. *P<0.05 vs. CTRL.

FIG. 8. Cardiac mitochondrial phospholipid fatty acid composition expressed as percentage of total fatty acids. Data are means of n=7-8/group. *P<0.001 vs. CTRL.

FIG. 9. Cardiac mitochondrial cardiolipin (CL) content for tetralinolyl CL(L4CL) and CL containing three linolyl and one arachidonic acid fatty acid groups (L3AA1) (molecular weights of 1448 and 1472, respectively) expressed as percentage of total CL. Data are means of n=8/group. *P<0.001 vs. CTRL.

FIGS. 10A-B. Effect of diet on the Ca²⁺ retention capacity. 10A: The fraction of preparations with the MPTP open plotted as a function of the cumulative amount of Ca²⁺ added to the cuvette containing isolated mitochondria. 10B: Mean Ca²⁺ infused to initiate MPTP opening. Data are means of n=8-9/group.

FIG. 11. Effect of different respiratory substrates on the Ca²⁺ retention capacity of CTRL mitochondria. Data are means of n=10-11/group. * p<0.05, palmitoylcarnitine+malate vs glutamate+malate. $ p<0.05, palmitoylcarnitine+malate vs succinate+rotenone. # p<0.05, palmitoylcarnitine+malate vs pyruvate+malate.

FIG. 12. Effect of DHA on mitochondrial Ca²⁺ retention capacity in the presence of different respiratory substrates. Data are means of n=9-11/group. * p<0.05, CTRL vs DHA.

FIG. 13. Effect of DHA on mitochondrial Ca²⁺ induced swelling. Ca²⁺ was added at time zero. Vehicle treated wells had stable absorbance over the 15 minutes of observation (data not show). Data are means of n=9/group.

FIG. 14. Effects of DHA on Ca²⁺-induced mitochondrial swelling as assessed from the relative change in absorbance from 0 to 15 minutes. Data are means of n=9/group. * p<0.05, CTRL vs DHA within same Ca²⁺ dose. # p<0.05, no Ca²⁺ vs 2 μmoles Ca^{2±}/mg mitochondrial protein. $ p<0.05, 2 μmoles Ca²⁺/mg mitochondrial protein vs 4 μmoles Ca²⁺/mg mitochondrial protein.

### DETAILED DESCRIPTION OF THE INVENTION

### I. Definitions

Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein when used in conjunction with the word “comprising,” the words “a” or “an” may mean one or more than one. As used herein “another” may mean at least a second or more. Furthermore, unless otherwise required by context, singular terms include pluralities and plural terms include the singular.

As used herein, “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, “preserve” and all its forms and tenses (including, for example, preserved, preserving, and preservation) refers to the maintenance of function or reduction in decline of function. For example, preserving mitochondrial function includes the maintenance of mitochondrial function or the reduction in decline of mitochondrial function (e.g., maintaining or increasing respiratory supercomplexes, maintaining or increasing oxidative phosphorylation, maintaining or decreasing MPTP opening, etc.).

As used herein, “treat” and all its forms and tenses (including, for example, treat, treating, treated, and treatment) refer to both therapeutic treatment and prophylactic or preventative treatment. Those in need of treatment include those already with a pathological condition of the invention (including, for example, heart failure or mitochondrial dysfunction) as well as those in which a pathological condition of the invention is to be prevented.

### II. The Present Invention

As detailed in several recent reviews, mitochondrial dysfunction and inefficient transfer of chemical to mechanical energy by the LV is a hallmark of cardiac pathology in HF, and is an attractive target for new therapies^{10, 11, 76, 77}. Globally, HF presents with an impaired transfer of energy from carbon substrates (fatty acid, glucose and lactate) and oxygen to ATP, and defects in subsequent ATP hydrolysis which drives contraction and relaxation^{10, 11}. In advanced HF there is a decrease in fatty acid oxidation and an increase in glucose oxidation, and impaired capacity for electron transport chain flux and oxidative phosphorylation^{76, 78-81}. Thus omega-3 PUFA might improve cardiac energetics and LV function through optimizing these parameters. Supplementation with DHA could prevent LV remodeling and dysfunction through modification of mitochondrial membrane and the function/structure of membrane proteins, and up-regulation of the expression of proteins through ligand activation of peroxisome proliferator-activated receptors (PPARs). While fatty acids are classically viewed as an energy substrate, they are also endogenous ligands for PPARs and regulate the expression of genes encoding key proteins controlling mitochondrial metabolism⁸²⁻⁸⁶. There is a variety of endogenous lipid ligands for PPARs, consisting primarily of long chain fatty acids. PPARα is expressed in heart, skeletal muscle, and liver, while PPARγ is expressed in adipose tissue. It is well established that dietary supplementation with EPA+DHA lowers plasma triglyceride and free fatty acid concentrations in a manner similar to PPARα and PPARγ agonists (fibrates (fenofibrate) and thioglitazones (rosiglitazone)), and thus reduces exposure of the heart to lipid substrates that could, in the long term, have toxic effects on the myocardium⁸⁷. Omega-3 PUFAs could also directly activate PPARα in the heart, and induce expression of key proteins involved in cardiac lipid metabolism, as they are activators of PPARα in vitro⁸⁸. This could prevent the deterioration of mitochondrial function and decrease in fatty acid oxidation that is classically observed in advanced HF, and thus might improve cardiac energetics and function through this mechanism, as recently suggested^{10, 76-81}.

Alternatively, supplementation with EPA+DHA could exert a protective effect through improvement in mitochondrial function and the efficiency of ATP generation. Rats fed fish oil high in EPA+DHA for 16 weeks showed a decrease in myocardial oxygen consumption (MVO2) without a decrease in LV power generation, resulting in greater LV mechanical efficiency in isolated perfused hearts⁵. This phenomenon was observed over a wide range of LV filling pressures and workloads. The mechanism(s) responsible for this effect is not clear, nor has this phenomenon been demonstrated in vivo. This is an important observation, as improvement in LV mechanical efficiency is considered a sound approach to improving LV mechanics in HF patients^{89, 91, 93, 94}.

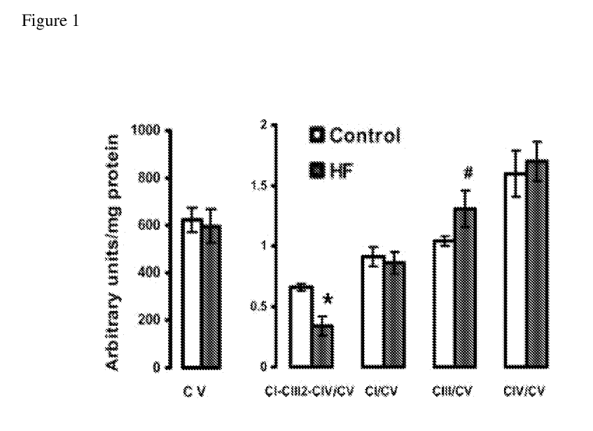

Mitochondria in the failing heart are characterized by normal mitochondrial volume density but a greater mitochondrial number and smaller size, and a lower capacity for respiration and oxidative phosphorylation^{13, 95-110}. An array of defects in ETC complexes has been noted in various forms of HF, with no consistent pattern^{46, 106, 107, 108, 111-116}. A comprehensive examination of cardiac mitochondrial function in HF was recently performed, with mitochondria isolated from the left ventricle of dogs with coronary micro-embolization induced HF of moderate severity (LV ejection fraction of 28%)¹. Oxidative phosphorylation was assessed as the integrative function of mitochondria, using a comprehensive variety of substrates in order to investigate mitochondrial membrane transport, dehydrogenase activity and ETC to oxidative phosphorylation. The supramolecular organization of the mitochondrial ETC also was investigated by native gel electrophoresis. There was a dramatic ˜40%-50% decrease in ADP-stimulated respiration with a variety of substrates that was not relieved by an uncoupler¹. Specifically, State 3 respiratory rates of both subsarcolemmal and interfibrillar mitochondria were significantly decreased with glutamate, pyruvate, or succinate plus rotenone as substrates, or with artificial electron donors. The P/O ratio and State IV respiration rates were normal in mitochondria from HF dogs, indicating no defects in the phosphorylation apparatus or uncoupling. While this suggests a defect in oxidative phosphorylation within the ETC, the individual activities of ETC complexes were normal, as were the activities of Krebs cycle enzymes^{1, 117, 118}. Importantly, the amount of the supercomplexes consisting of complex I/complex III dimer/complex IV, the major form of the respirasome essential for oxidative phosphorylation was decreased (FIG. 1). This demonstrates that a mitochondrial defect in HF lies in the supermolecular assembly rather than in the individual components of the ETC1.

Formation of respiratory supercomplexes requires cardiolipin (CL), an inner membrane tetra-acyl phospholipid comprised primarily of linoleic acid (18:2n6) that anchors cytochrome C to the membrane and prevents apoptosis¹⁵⁻²³. Depletion of CL has been noted in HF, and a recent study showed that a high linoleic acid diet restored CL and L4CL, improved LV function, and prolonged survival in aged spontaneously hypertensive heart failure rats¹²²⁻¹²⁵. This suggests that depletion in CL contributes to LV dysfunction and early death in HF. As noted above, a major defect in mitochondria in HF is decreased formation of respiratory supercomplexes, thus restoration of normal levels of supercomplexes should improve energy transfer and LV function in HF. Without being bound by theory, increasing the content of CL and/or L4CL in cardiac mitochondria improves outcome in HF by preventing MPTP and increasing supercomplex levels, improving ETC flux and oxidative phosphorylation, decreasing cardiomyocyte death and improving LV function and survival. Pepe et al. showed that treatment with fish oil high in EPA+DHA for 6 weeks was shown to increase total CL content in cardiac mitochondria in 24 month old rats by 40%¹²⁶. McMillin et al observed a similar effect in dogs with 60 weeks of treatment, showing a 54% increase in total CL in cardiac mitochondria¹²⁷. The effect of EPA+DHA on the composition of fatty acyl moieties of CL has not been reported, though in our studies we observed that dietary supplementation with EPA and DHA increased both total CL and L4CL content, and resulted in an increase in DHA incorporation into CL, without significant EPA incorporation. Based on these findings, we assessed the effects of omega-3 PUFA supplementation on mitochondrial CL content and composition, respiratory supercomplexes, oxidative phosphorylation, and progression of HF. In particular aspects, the invention is drawn to administering DHA alone for treating HF wherein DHA alone has the effect of: preserving cardiac mitochondrial function, maintaining cardiac respiratory supercomplexes, increasing cardiac respiratory supercomplexes, maintaining cardiac oxidative phosphorylation, increasing cardiac oxidative phosphorylation, preventing cardiac MPTP opening, maintaining cardiac levels of DHA, increasing cardiac levels of DHA, maintaining levels of EPA, increasing levels of EPA, maintaining levels of cardiac mitochondrial cardiolipin, increasing levels of cardiac mitochondrial cardiolipin, inhibiting cardiomyocyte death by apoptosis and inhibiting cardiomyocyte death by necrosis. Alternatively, in particular aspects the treatment contributes to one or more clinical end points selected from the group consisting of reduced left ventricular volume, improved cardiac contractile function, reduced cardiac-related hospitalization, reduced cardiac-medical complications, improved quality of life and reduced mortality.

Normal function of cardiac mitochondria is important not only for maintaining sufficient ATP generation and contractile function, but also for prevention of apoptosis and/or necrosis, and loss of cardiomyocytes. Formation of the mitochondrial permeability transition pore (MPTP) in cardiac mitochondria is strongly associated with cardiomyocyte death, tissue injury, development and progression of HF, and poor contractile recovery with ischemia/reperfusion stress^{12, 128-131}. The MPTP is a large diameter (3 nm), high conductance, voltage-dependent channel that allows passage of water, ions, and molecules up to ˜1.5 kD^{129, 131, 132}. High extra-mitochondrial Ca²⁺ triggers MPTP opening and Ca²⁺ chelation causes it to rapidly close. As recently reviewed, there is growing evidence to suggest that pharmacological targeting of the MPTP would be beneficial for preventing or reversing the progression of HF^{128, 132, 134}. In genetic models of hypertrophic HF there is greater MPTP opening in response to Ca²⁺ stress, and there is evidence to suggest that formation of the MPTP is a key component of the pathological processes caused by adrenergic overdrive and Ca²⁺ overload in HF^{99, 135-137}. Unlike healthy cardiomyocytes, cells from dogs with HF showed MPTP opening without ischemia or calcium overload, which was attenuated by cyclosporin A, suggesting that MPTP opening is partially responsible for the mitochondrial dysfunction described in HF, and trigger cell death¹³⁸.

Cardiomyocyte death by apoptosis or necrosis is elevated in HF and contributes to progressive LV dysfunction and remodeling, and thus prevention of MPTP and subsequent triggering of death is considered a primary target for HF therapy¹³⁸⁻¹⁴⁹. While the molecular components and structure of the MPTP are not precisely known, there is evidence to suggest that it is affected by the phospholipid composition of mitochondrial membranes and the assembly of proteins within the membrane^{12, 18, 128-130, 132}. The fatty acid composition of dietary lipid affects mitochondrial function and cardiomyocyte apoptosis, with more apoptosis with saturated fatty acids, and less with unsaturated fatty acids¹⁵⁰⁻¹⁵⁹. We found that there is a lower number of apoptotic cardiomyocytes with a high fat diet rich in linoleic acid (18:2n-6) compared to a high saturated fat diet in normal rats, and also less apoptosis with supplementation with either ALA (from flaxseed oil) or EPA+DHA in rats with aortic banding^{2, 150}. The fatty acyl moiety of CL is comprised mostly of 18:2n-6, with ˜50-80% being tetralinoleoyl CL (L4CL)^{123-125, 160}. Substitution with other long chain fatty acids (particularly 16:0, 18:0 or 18:1), impairs mitochondrial function^{124, 125, 161-166}. HF and cardiac hypertrophy deplete CL, decrease L4CL, and increase saturated fatty acyl moieties in CL¹⁶⁴, which is partially prevented by high dietary 18:2n-6¹⁶⁷⁻¹⁶⁹. These effects may be due to changes in mitochondrial membrane content of CL, as cytochrome c is anchored to the inner mitochondrial membrane by CL¹⁷⁰. Thus an increase in L4CL or total CL content should prevent the release of cytochrome c, MPTP formation, and subsequent cardiomyocyte apoptosis and cell death^{15, 16, 171}.

Consumption of omega-3 PUFA is associated with reduced inflammation, as reflected in the inverse relationship with circulating tumor necrosis factor alpha (TNFα), TNFβ, and interleukin (IL) 1β and IL6^{28, 66, 71, 186-192}. Emerging evidence suggests that chronic up-regulation of stress-activated cytokines both systemically and in myocardium contributes to the progression of HF¹⁹³. On the other hand, direct cytokine blockade with the soluble tumor necrosis factor antagonist ETANERCEPT was not effective in HF patients¹⁹⁴⁻¹⁹⁷. This has led to the postulation that broader indirect anti-inflammatory approaches, such as treatment with statins or pentoxifylline, may be useful for treating HF¹⁹⁸⁻²⁰⁴. We recently observed a dramatic decrease in serum TNFα in both sham and aortic banded rats treated with EPA+DHA², which is similar to observations made in humans^{28, 70, 71}. In addition, EPA+DHA lowers prostaglandin-mediated inflammation by reducing arachidonic acid incorporation into membrane phospholipids, as seen in a decrease in urinary output of the main thromboxane metabolite in rats treated with EPA+DHA. While a focus of the invention is on the effects of omega-3 PUFA supplementation for treating HF and mitochondria dysfunction (esp. the effect of administering DHA alone), we will also explore the effects on inflammatory markers in all studies.

As discussed in the Brief Description of the Invention above, the present invention is drawn, inter alia, to methods that result in the improvement of cardiac function. Particular embodiments include: (i) methods of treating heart failure in a subject, (ii) methods of increasing cardiac mitochondrial cardiolipin in a subject, (iii) methods of suppressing cardiac MPTP opening in a subject, and (iv) methods of treating cardiac mitochondrial dysfunction in a subject.

The skilled artisan will understand that heart failure can be treated through a number of different means that are well known in the literature and in practice. Particular examples include preserving cardiac mitochondrial function, maintaining cardiac respiratory supercomplexes, increasing cardiac respiratory supercomplexes, maintaining cardiac oxidative phosphorylation, increasing cardiac oxidative phosphorylation, preventing cardiac MPTP opening, maintaining cardiac levels of DHA, increasing cardiac levels of DHA, maintaining levels of EPA, increasing levels of EPA, maintaining levels of cardiac mitochondrial cardiolipin, increasing levels of cardiac mitochondrial cardiolipin, inhibiting cardiomyocyte death by apoptosis and inhibiting cardiomyocyte death by necrosis. The skilled artisan will also understand that due to the difficulty in assessing the results of treatment means directed to cellular or molecular changes, clinical end points may be used as a goal of treatment. Accordingly, in particular aspects of the invention treatment contributes to one or more clinical end points selected from the group consisting of reduced left ventricular volume, improved cardiac contractile function, reduced cardiac-related hospitalization, reduced cardiac-medical complications, improved quality of life and reduced mortality.

In each of these embodiments, a pharmaceutical composition comprising, consisting essentially of, or consisting of a therapeutically effective amount of DHA, or DHA and EPA, is administered to a subject in need of treatment.

In each of these embodiments, the subject is a human, a non-human primate, bird, horse, cow, goat, sheep, a companion animal, such as a dog, cat or rodent, or other mammal.

The present invention is also directed: (i) methods of preventing heart failure in a subject, using the same methodologies described herein for methods of treating heart failure in a subject.

### III. Formulations and Doses

In certain aspects of the invention drawn to compositions for treating heart failure or preserving mitochondria, the compositions are formulated to achieve this end. Generally, the composition comprises, consists of, or consists essentially of, an omega-3 PUFA, wherein the omega-3 PUFA is ALA, EPA, DHA, or any combination thereof. In further particular aspects, the omega-3 PUFA is DHA. DHA or the other omega-3 PUFA can be esterified (e.g., as either a triglyceride or an ethyl ester), and can be in liquid form or capsule form (see, for example, U.S. Pat. No. 7,041,324, which is incorporated herein in its entirety).

As is well known in the medical arts, dosages for any one subject depends upon many factors, including patient size, body surface area, age, the particular molecule or composition to be administered, sex, time, route of administration, general health, and the presence of other molecules or compositions being administered concurrently. The compositions of the invention may be administered locally or systemically.

Without being bound by theory, in particular aspects of the invention it is contemplated that the therapeutically effective amount of DHA, EPA, or ALA, used in the methods of the invention ranges from about 0.1 and 10 g/day, about 0.1 to 9.9 g/day, about 0.1 to 9.8 g/day, about 0.1 to 9.7 g/day, about 0.1 to 9.6 g/day, about 0.1 to 9.5 g/day, about 0.1 to 9.4 g/day, about 0.1 to 9.3 g/day, about 0.1 to 9.2 g/day, about 0.1 to 9.1 g/day, about 0.1 to 9.0 g/day, about 0.1 to 8.9 g/day, about 0.1 to 8.8 g/day, about 0.1 to 8.7 g/day, about 0.1 to 8.6 g/day, about 0.1 to 8.5 g/day, about 0.1 to 8.4 g/day, about 0.1 to 8.3 g/day, about 0.1 to 8.2 g/day, about 0.1 to 8.1 g/day, about 0.1 to 8.0 g/day, about 0.1 to 7.9 g/day, about 0.1 to 7.8 g/day, about 0.1 to 7.7 g/day, about 0.1 to 7.6 g/day, about 0.1 to 7.5 g/day, about 0.1 to 7.4 g/day, about 0.1 to 7.3 g/day, about 0.1 to 7.2 g/day, about 0.1 to 7.1 g/day, about 0.1 to 7.0 g/day, about 0.1 to 6.9 g/day, about 0.1 to 6.8 g/day, about 0.1 to 6.7 g/day, about 0.1 to 6.6 g/day, about 0.1 to 6.5 g/day, about 0.1 to 6.4 g/day, about 0.1 to 6.3 g/day, about 0.1 to 6.2 g/day, about 0.1 to 6.1 g/day, about 0.1 to 6.0 g/day, about 0.1 to 5.9 g/day, about 0.1 to 5.8 g/day, about 0.1 to 5.7 g/day, about 0.1 to 5.6 g/day, about 0.1 to 5.5 g/day, about 0.1 to 5.4 g/day, about 0.1 to 5.3 g/day, about 0.1 to 5.2 g/day, about 0.1 to 5.1 g/day, about 0.1 to 5.0 g/day, about 0.1 to 4.9 g/day, about 0.1 to 4.8 g/day, about 0.1 to 4.7 g/day, about 0.1 to 4.6 g/day, about 0.1 to 4.5 g/day, about 0.1 to 4.4 g/day, about 0.1 to 4.3 g/day, about 0.1 to 4.2 g/day, about 0.1 to 4.1 g/day, about 0.1 to 4.0 g/day, about 0.1 to 3.9 g/day, about 0.1 to 3.8 g/day, about 0.1 to 3.7 g/day, about 0.1 to 3.6 g/day, about 0.1 to 3.5 g/day, about 0.1 to 3.4 g/day, about 0.1 to 3.3 g/day, about 0.1 to 3.2 g/day, about 0.1 to 3.1 g/day, about 0.1 to 3.0 g/day, about 0.1 to 2.9 g/day, about 0.1 to 2.8 g/day, about 0.1 to 2.7 g/day, about 0.1 to 2.6 g/day, about 0.1 to 2.5 g/day, about 0.1 to 2.4 g/day, about 0.1 to 2.3 g/day, about 0.1 to 2.2 g/day, about 0.1 to 2.1 g/day, about 0.1 to 2.0 g/day, about 0.1 to 1.9 g/day, about 0.1 to 1.8 g/day, about 0.1 to 1.7 g/day, about 0.1 to 1.6 g/day, about 0.1 to 1.5 g/day, about 0.1 to 1.4 g/day, about 0.1 to 1.3 g/day, about 0.1 to 1.2 g/day, about 0.1 to 1.1 g/day, and about 0.1 to 1.0 g/day.

Without being bound by theory, in particular aspects of the invention it is contemplated that the therapeutically effective amount of DHA, EPA, or ALA, used in the methods of the invention, ranges from about 0.5 to 5 g/day, about 0.5 to 4.9 g/day, about 0.5 to 4.8 g/day, about 0.5 to 4.7 g/day, about 0.5 to 4.6 g/day, about 0.5 to 4.4 g/day, about 0.5 to 4.3 g/day, about 0.5 to 4.2 g/day, about 0.5 to 4.1 g/day, about 0.5 to 4.0 g/day, 0.6 to 5 g/day, about 0.6 to 4.9 g/day, about 0.6 to 4.8 g/day, about 0.6 to 4.7 g/day, about 0.6 to 4.6 g/day, about 0.6 to about 4.5, about 0.6 to 4.4 g/day, about 0.6 to 4.3 g/day, about 0.6 to 4.2 g/day, about 0.6 to 4.1 g/day, about 0.6 to 4.0 g/day, 0.7 to 5 g/day, about 0.7 to 4.9 g/day, about 0.7 to 4.8 g/day, about 0.7 to 4.7 g/day, about 0.7 to 4.6 g/day, about 0.7 to about 4.5, about 0.7 to 4.4 g/day, about 0.7 to 4.3 g/day, about 0.7 to 4.2 g/day, about 0.7 to 4.1 g/day, about 0.7 to 4.0 g/day, 0.8 to 5 g/day, about 0.8 to 4.9 g/day, about 0.8 to 4.8 g/day, about 0.8 to 4.7 g/day, about 0.8 to 4.6 g/day, about 0.8 to about 4.5, about 0.8 to 4.4 g/day, about 0.8 to 4.3 g/day, about 0.8 to 4.2 g/day, about 0.8 to 4.1 g/day, about 0.8 to 4.0 g/day, 0.9 to 5 g/day, about 0.9 to 4.9 g/day, about 0.9 to 4.8 g/day, about 0.9 to 4.7 g/day, about 0.9 to 4.6 g/day, about 0.9 to about 4.5, about 0.9 to 4.4 g/day, about 0.9 to 4.3 g/day, about 0.9 to 4.2 g/day, about 0.9 to 4.1 g/day, about 0.9 to 4.0 g/day, 1 to 5 g/day, about 1 to 4.9 g/day, about 1 to 4.8 g/day, about 1 to 4.7 g/day, about 1 to 4.6 g/day, about 1 to about 4.5, about 1 to 4.4 g/day, about 1 to 4.3 g/day, about 1 to 4.2 g/day, about 1 to 4.1 g/day, and about 1 to 4.0 g/day.

Without being bound by theory, in particular aspects of the invention it is contemplated that the therapeutically effective amount of DHA, EPA, or ALA, used in the methods of the invention, is about 0.5 g/day, about 0.6 g/day, 0.7 g/day, 0.8 g/day, 0.9 g/day, 1.0 g/day, 1.1 g/day, 1.2 g/day, 1.3 g/day, 1.4 g/day, 1.5 g/day, 1.6 g/day, 1.7 g/day, 1.8 g/day, 1.9 g/day, 2.0 g/day, 2.0 g/day, 2.1 g/day, 2.2 g/day, 2.3 g/day, 2.4 g/day, 2.5 g/day, 2.6 g/day, 2.7 g/day, 2.8 g/day, 2.9 g/day, 3.0 g/day, 3.0 g/day, 3.1 g/day, 3.2 g/day, 3.3 g/day, 3.4 g/day, 3.5 g/day, 3.6 g/day, 3.7 g/day, 3.8 g/day, 3.9 g/day, 4.0 g/day, 4.0 g/day, 4.1 g/day, 4.2 g/day, 4.3 g/day, 4.4 g/day, 4.5 g/day, 4.6 g/day, 4.7 g/day, 4.8 g/day, 4.9 g/day, 5.0 g/day, 5.0 g/day, 5.1 g/day, 5.2 g/day, 5.3 g/day, 5.4 g/day, 5.5 g/day, 5.6 g/day, 5.7 g/day, 5.8 g/day, 5.9 g/day, 6.0 g/day, 6.0 g/day, 6.1 g/day, 6.2 g/day, 6.3 g/day, 6.4 g/day, 6.5 g/day, 6.6 g/day, 6.7 g/day, 6.8 g/day, 6.9 g/day, 7.0 g/day, 8.0 g/day, 8.1 g/day, 8.2 g/day, 8.3 g/day, 8.4 g/day, 8.5 g/day, 8.6 g/day, 8.7 g/day, 8.8 g/day, 8.9 g/day, 9.0 g/day, 9.0 g/day, 9.1 g/day, 9.2 g/day, 9.3 g/day, 9.4 g/day, 9.5 g/day, 9.6 g/day, 9.7 g/day, 9.8 g/day, 9.9 g/day, and 10.0 g/day.

In preferred aspects of the invention where the pharmaceutical composition comprises both EPA and DHA, the ratio of EPA to DHA is between about 1:100 and about 1:4 by weight, more preferably between about 1:49 and about 1:9 by weight. In a particular example, the ratio of EPA to DHA in the pharmaceutical composition is about 0.1:1 by weight.

In preferred aspects, the pharmaceutical composition used in the relevant methods of the present invention comprises between about 0.5 g and about 5 g of DHA, and between about 0.01 g and about 0.5 g of EPA, more preferably the pharmaceutical composition comprises between about 0.5 g and about 5 g of DHA, and between about 0.01 g and about 0.1 g of EPA.

In particular aspects of the invention it is contemplated that a pharmaceutical composition comprising DHA alone as the active ingredient (or, a composition consisting of DHA or consisting essentially of DHA) is administered to or otherwise provided to a subject in need thereof in accordance with the invention as described herein, wherein the therapeutically effective amount of DHA ranges from about 0.1 and 10 g/day, about 0.1 to 9.9 g/day, about 0.1 to 9.8 g/day, about 0.1 to 9.7 g/day, about 0.1 to 9.6 g/day, about 0.1 to 9.5 g/day, about 0.1 to 9.4 g/day, about 0.1 to 9.3 g/day, about 0.1 to 9.2 g/day, about 0.1 to 9.1 g/day, about 0.1 to 9.0 g/day, about 0.1 to 8.9 g/day, about 0.1 to 8.8 g/day, about 0.1 to 8.7 g/day, about 0.1 to 8.6 g/day, about 0.1 to 8.5 g/day, about 0.1 to 8.4 g/day, about 0.1 to 8.3 g/day, about 0.1 to 8.2 g/day, about 0.1 to 8.1 g/day, about 0.1 to 8.0 g/day, about 0.1 to 7.9 g/day, about 0.1 to 7.8 g/day, about 0.1 to 7.7 g/day, about 0.1 to 7.6 g/day, about 0.1 to 7.5 g/day, about 0.1 to 7.4 g/day, about 0.1 to 7.3 g/day, about 0.1 to 7.2 g/day, about 0.1 to 7.1 g/day, about 0.1 to 7.0 g/day, about 0.1 to 6.9 g/day, about 0.1 to 6.8 g/day, about 0.1 to 6.7 g/day, about 0.1 to 6.6 g/day, about 0.1 to 6.5 g/day, about 0.1 to 6.4 g/day, about 0.1 to 6.3 g/day, about 0.1 to 6.2 g/day, about 0.1 to 6.1 g/day, about 0.1 to 6.0 g/day, about 0.1 to 5.9 g/day, about 0.1 to 5.8 g/day, about 0.1 to 5.7 g/day, about 0.1 to 5.6 g/day, about 0.1 to 5.5 g/day, about 0.1 to 5.4 g/day, about 0.1 to 5.3 g/day, about 0.1 to 5.2 g/day, about 0.1 to 5.1 g/day, about 0.1 to 5.0 g/day, about 0.1 to 4.9 g/day, about 0.1 to 4.8 g/day, about 0.1 to 4.7 g/day, about 0.1 to 4.6 g/day, about 0.1 to 4.5 g/day, about 0.1 to 4.4 g/day, about 0.1 to 4.3 g/day, about 0.1 to 4.2 g/day, about 0.1 to 4.1 g/day, about 0.1 to 4.0 g/day, about 0.1 to 3.9 g/day, about 0.1 to 3.8 g/day, about 0.1 to 3.7 g/day, about 0.1 to 3.6 g/day, about 0.1 to 3.5 g/day, about 0.1 to 3.4 g/day, about 0.1 to 3.3 g/day, about 0.1 to 3.2 g/day, about 0.1 to 3.1 g/day, about 0.1 to 3.0 g/day, about 0.1 to 2.9 g/day, about 0.1 to 2.8 g/day, about 0.1 to 2.7 g/day, about 0.1 to 2.6 g/day, about 0.1 to 2.5 g/day, about 0.1 to 2.4 g/day, about 0.1 to 2.3 g/day, about 0.1 to 2.2 g/day, about 0.1 to 2.1 g/day, about 0.1 to 2.0 g/day, about 0.1 to 1.9 g/day, about 0.1 to 1.8 g/day, about 0.1 to 1.7 g/day, about 0.1 to 1.6 g/day, about 0.1 to 1.5 g/day, about 0.1 to 1.4 g/day, about 0.1 to 1.3 g/day, about 0.1 to 1.2 g/day, about 0.1 to 1.1 g/day, and about 0.1 to 1.0 g/day.

In other particular aspects of the invention it is contemplated that a composition of DHA alone (i.e., a composition consisting of DHA or consisting essentially of DHA) is administered to or otherwise provided to a subject in need thereof in accordance with the invention as described herein, wherein the therapeutically effective amount of DHA ranges from about 0.5 to 5 g/day, about 0.5 to 4.9 g/day, about 0.5 to 4.8 g/day, about 0.5 to 4.7 g/day, about 0.5 to 4.6 g/day, about 0.5 to 4.4 g/day, about 0.5 to 4.3 g/day, about 0.5 to 4.2 g/day, about 0.5 to 4.1 g/day, about 0.5 to 4.0 g/day, 0.6 to 5 g/day, about 0.6 to 4.9 g/day, about 0.6 to 4.8 g/day, about 0.6 to 4.7 g/day, about 0.6 to 4.6 g/day, about 0.6 to about 4.5, about 0.6 to 4.4 g/day, about 0.6 to 4.3 g/day, about 0.6 to 4.2 g/day, about 0.6 to 4.1 g/day, about 0.6 to 4.0 g/day, 0.7 to 5 g/day, about 0.7 to 4.9 g/day, about 0.7 to 4.8 g/day, about 0.7 to 4.7 g/day, about 0.7 to 4.6 g/day, about 0.7 to about 4.5, about 0.7 to 4.4 g/day, about 0.7 to 4.3 g/day, about 0.7 to 4.2 g/day, about 0.7 to 4.1 g/day, about 0.7 to 4.0 g/day, 0.8 to 5 g/day, about 0.8 to 4.9 g/day, about 0.8 to 4.8 g/day, about 0.8 to 4.7 g/day, about 0.8 to 4.6 g/day, about 0.8 to about 4.5, about 0.8 to 4.4 g/day, about 0.8 to 4.3 g/day, about 0.8 to 4.2 g/day, about 0.8 to 4.1 g/day, about 0.8 to 4.0 g/day, 0.9 to 5 g/day, about 0.9 to 4.9 g/day, about 0.9 to 4.8 g/day, about 0.9 to 4.7 g/day, about 0.9 to 4.6 g/day, about 0.9 to about 4.5, about 0.9 to 4.4 g/day, about 0.9 to 4.3 g/day, about 0.9 to 4.2 g/day, about 0.9 to 4.1 g/day, about 0.9 to 4.0 g/day, 1 to 5 g/day, about 1 to 4.9 g/day, about 1 to 4.8 g/day, about 1 to 4.7 g/day, about 1 to 4.6 g/day, about 1 to about 4.5, about 1 to 4.4 g/day, about 1 to 4.3 g/day, about 1 to 4.2 g/day, about 1 to 4.1 g/day, and about 1 to 4.0 g/day.

In other particular aspects of the invention it is contemplated that a composition of DHA alone (i.e., a composition consisting of DHA or consisting essentially of DHA) is administered to or otherwise provided to a subject in need thereof in accordance with the invention as described herein, wherein the therapeutically effective amount of DHA is about 0.5 g/day, about 0.6 g/day, 0.7 g/day, 0.8 g/day, 0.9 g/day, 1.0 g/day, 1.1 g/day, 1.2 g/day, 1.3 g/day, 1.4 g/day, 1.5 g/day, 1.6 g/day, 1.7 g/day, 1.8 g/day, 1.9 g/day, 2.0 g/day, 2.0 g/day, 2.1 g/day, 2.2 g/day, 2.3 g/day, 2.4 g/day, 2.5 g/day, 2.6 g/day, 2.7 g/day, 2.8 g/day, 2.9 g/day, 3.0 g/day, 3.0 g/day, 3.1 g/day, 3.2 g/day, 3.3 g/day, 3.4 g/day, 3.5 g/day, 3.6 g/day, 3.7 g/day, 3.8 g/day, 3.9 g/day, 4.0 g/day, 4.0 g/day, 4.1 g/day, 4.2 g/day, 4.3 g/day, 4.4 g/day, 4.5 g/day, 4.6 g/day, 4.7 g/day, 4.8 g/day, 4.9 g/day, 5.0 g/day, 5.0 g/day, 5.1 g/day, 5.2 g/day, 5.3 g/day, 5.4 g/day, 5.5 g/day, 5.6 g/day, 5.7 g/day, 5.8 g/day, 5.9 g/day, 6.0 g/day, 6.0 g/day, 6.1 g/day, 6.2 g/day, 6.3 g/day, 6.4 g/day, 6.5 g/day, 6.6 g/day, 6.7 g/day, 6.8 g/day, 6.9 g/day, 7.0 g/day, 8.0 g/day, 8.1 g/day, 8.2 g/day, 8.3 g/day, 8.4 g/day, 8.5 g/day, 8.6 g/day, 8.7 g/day, 8.8 g/day, 8.9 g/day, 9.0 g/day, 9.0 g/day, 9.1 g/day, 9.2 g/day, 9.3 g/day, 9.4 g/day, 9.5 g/day, 9.6 g/day, 9.7 g/day, 9.8 g/day, 9.9 g/day, and 10.0 g/day. In preferred aspects, the therapeutically effective amount of DHA in the pharmaceutical composition is between about 0.1 g and about 5.0 g, more preferably between about 1 g and about 4 g. In a particular example, the therapeutically effective amount of DHA in the pharmaceutical composition is 2.5 g, 3.0 g or 3.5 g.

Administration frequencies for the pharmaceutical compositions of the present invention include 4, 3, 2 or once daily, every other day, every third day, every fourth day, every fifth day, every sixth day, once weekly, every eight days, every nine days, every ten days, bi-weekly, monthly and bi-monthly. In preferred aspects, the pharmaceutical composition is administered orally once daily. The duration of treatment will be based on the condition being treated and will be best determined by the attending physician. However, continuation of treatment is contemplated to last for a number of days, weeks, months or years. Indeed, in some instances, treatment may continue for the entire life of the subject.

The pharmaceutical compositions of the present invention may be formulated, for example, for oral, sublingual, intranasal, intraocular, rectal, transdermal, mucosal, pulmonary, topical or parenteral administration. Parenteral modes of administration include without limitation, intradermal, subcutaneous (s.c., s.q., sub-Q, Hypo), intramuscular (i.m.), intravenous (i.v.), intraperitoneal (i.p.), intra-arterial, intramedulary, intracardiac, intra-articular (joint), intrasynovial (joint fluid area), intracranial, intraspinal, and intrathecal (spinal fluids). Any known device useful for parenteral injection or infusion of drug formulations can be used to effect such administration. In preferred aspects of each of the embodiments on the invention, the pharmaceutical composition is administered to the subject as an oral formulation.

The pharmaceutical compositions of the present invention will comprise one or more omega-3 PUFAs as described herein, and each omega-3 PUFA present in the pharmaceutical compositions can independently be esterified. The pharmaceutical compositions comprising omega-3 PUFAs may also be comprised of one or more carrier, diluent and excipient.

Depending on the means of administration, the dosage may be administered all at once, such as with an oral formulation in a capsule or liquid, or slowly over a period of time, such as with an intramuscular or intravenous administration.

### IV. Examples

Although the examples described herein are described with reference to EPA+DHA, DHA is used with superior results. A surprising and unexpected result is that DHA alone is more effective for treating HF and preserving or improving mitochondrial function. In particular aspects, DHA increases mitochondrial content of DHA, EPA and CL, which results in reduction of progression of HF and preservation of mitochondria in a subject experiencing HF.

### Example 1

### LV Dysfunction is Prevented by EPA+DHA Supplementation in Pressure Overload

A dose-response study was completed with EPA+DHA from fish oil using the rat abdominal aortic constriction pressure overload model of HF. In this model a permanent band is tied around the supra-renal abdominal aorta of male Wistar rats (˜200 g) by placing a blunt needle (20G) along the aorta and tying a 3-0 silk suture around both the aorta and the needle. The needle is removed, leaving the diameter of the aortic lumen determined by the diameter of the needle. The increase in aortic pressure results in progressive LV hypertrophy, mitochondrial dysfunction and decreased activity of mitochondrial enzymes, and LV dilation and contractile dysfunction and thus HF^{2, 207-213}.

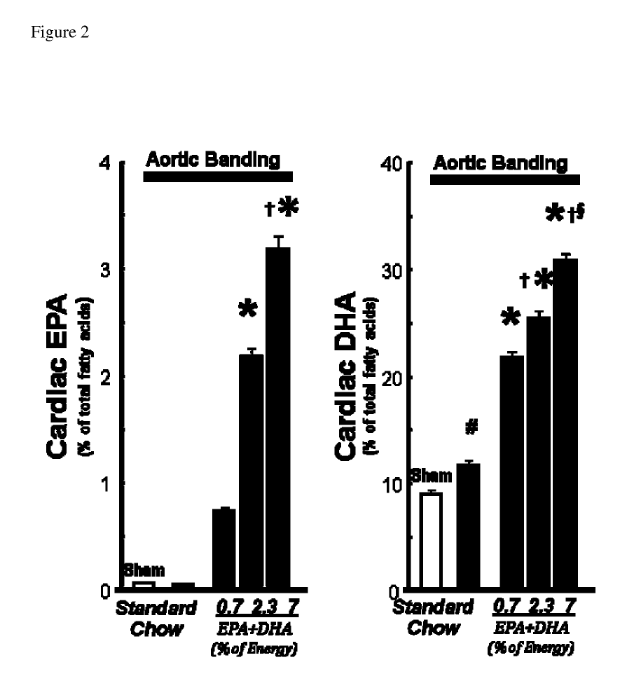

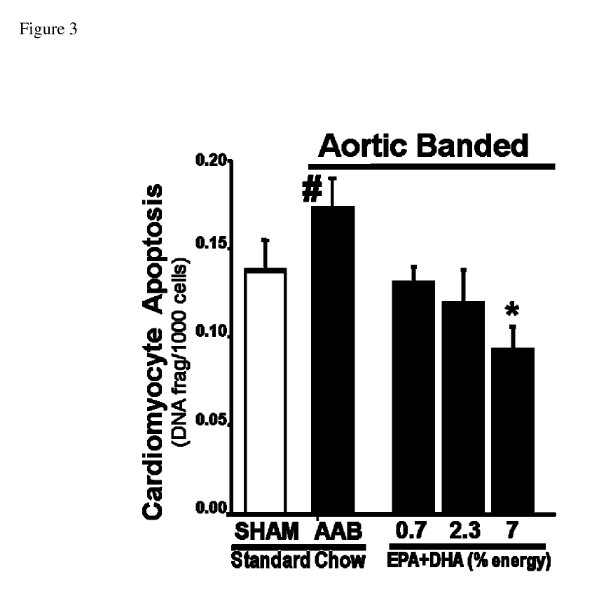

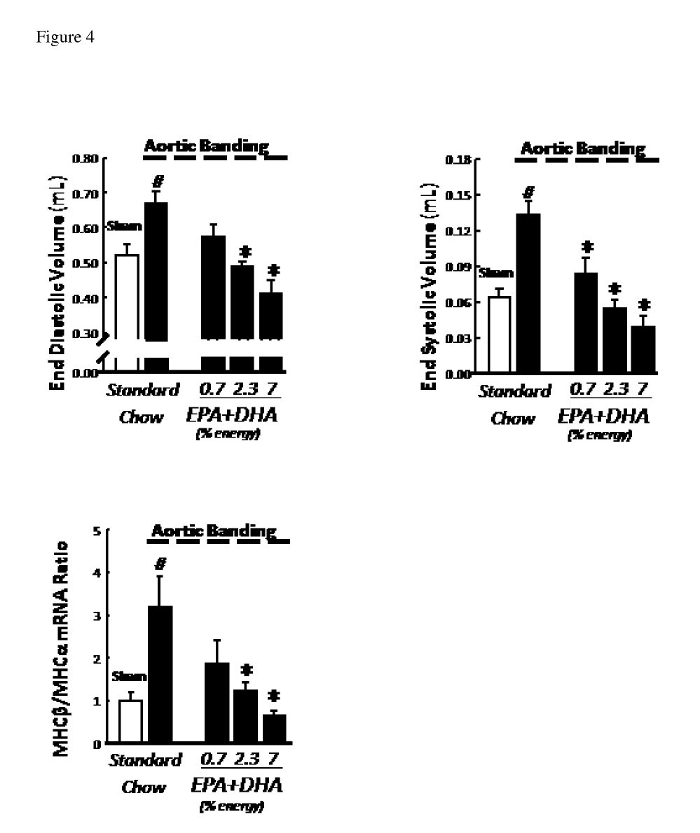

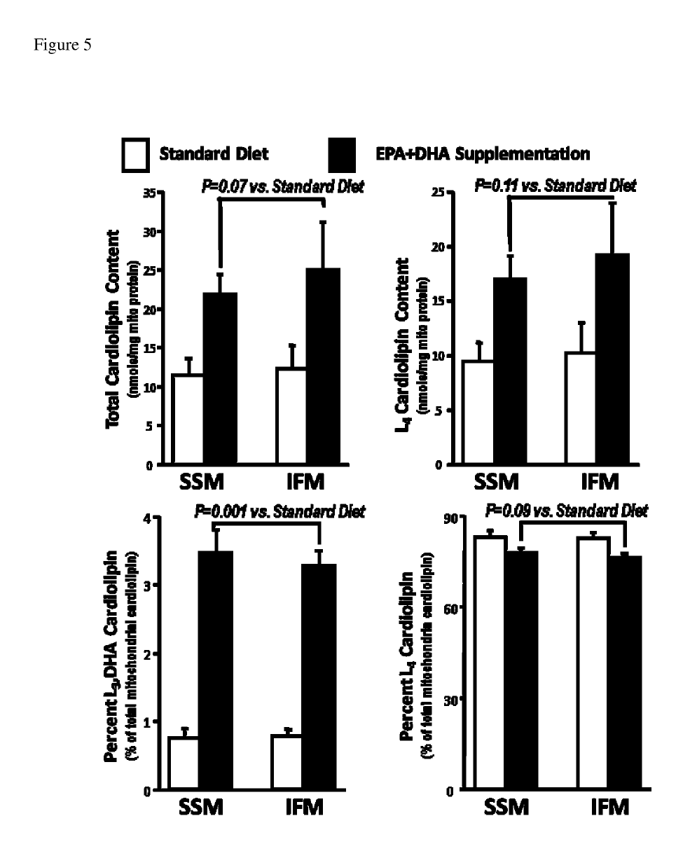

Rats were subjected to abdominal aortic banding and assigned them to 12 wks of treatment with either a standard chow or chow supplemented with EPA+DHA from fish oil at either 0.7, 2.3 or 7% of the total energy intake, with an EPA/DHA ration at a 30/70 mix, respectively. These doses correspond to estimated human doses of 1.6, 5.1 and 15.6 g/day (calculated assuming an energy intake of 2000 kcals/day in humans, and 9 kcals/g of EPA+DHA), thus we spanned both sides of the FDA approved human dose of 3.4 g/day. The EPA and DHA content of cardiac phospholipids was measure by gas chromatography, and showed a dose-dependent increase in both EPA and DHA (FIG. 2)⁵⁵. There were no differences in body mass or heart rate among groups. LV mass was 37% greater in the banded rats compared to sham on the standard diet. A similar degree of LV hypertrophy was observed in banded rats treated with EPA+DHA, thus treatment did not reduce cardiac growth in response to pressure overload. Cardiomyocyte apoptosis was measured in frozen sections from the mid LV free wall using TdT-mediated dUTP Nick-End Labeling, with ventricular anti-myosin antibody to identify cardiomyocytes as previously described^{147, 214}. Aortic banding increased cardiomyocyte apoptosis compared to sham rats on the standard chow, but not in the banded rats fed EPA+DHA. Apoptosis was decreased in the banded rats at the highest dose of EPA+DHA compared to standard chow sham rats (FIG. 3). On standard chow there was LV chamber enlargement compared to sham operated rats, which was prevented in a dose-dependent manner by EPA+DHA (FIG. 4). LV remodeling and dysfunction in response to aortic banding in rats fed the standard chow was associated with a significant increase in the mRNA expression for myosin heavy chain (MHC) β relative to MHCα, which was prevented by EPA+DHA (FIG. 4). These data are the first to demonstrate that supplementation with EPA+DHA prevents cardiomyocyte apoptosis and development of LV dysfunction and dilation.

### Example 2

### EPA+DHA Supplementation Increases Cardiolipin

Supplementation with EPA+DHA could improve mitochondrial function by increasing the content of CL in mitochondrial membranes. It has previously been shown that treatment with fish oil high in EPA+DHA increases total CL content in cardiac mitochondria in old rats by 40%¹²⁶ and in dogs by 54%¹²⁷. As discussed above, the fatty acyl moieties of CL are comprised primarily of linoleic acid (18:2n6), with most CL being tetralinoleoyl CL (L4CL) (˜50%-80%)¹²⁵. Depletion of CL or substitution of 18:2n6 with saturated or monounsaturated fatty acyl moieties impairs mitochondrial function¹²⁵. A high level of CL in mitochondrial membranes is needed for formation of respiratory supercomplexes^{20, 21}. CL also prevents apoptosis and is required for normal mitochondrial function^{15, 16}. In some rodent models of HF there is depletion of total CL and L4CL and an increase in saturated fatty acyl moieties in CL¹²⁵. We completed a pilot study to assess the effects of EPA+DHA supplementation on CL content and fatty acyl composition in cardiac mitochondria. Normal healthy male rats were fed a standard lab chow or supplemented with EPA+DHA (2.3% of energy intake)(n=3/group) for 12 weeks. This dose corresponds to a human intake of ˜5 g/day of EPA+DHA (calculated assuming an energy intake of 2000 kcal/d in man), which is in the range of the currently approved dose of EPA+DHA for the treatment of hypertriglyceridaemia (3.4 g/day). Two populations of cardiac mitochondria (subsarcolemmal (SSM) and intrafibrillar (IFM)) were isolated as described in our recent studies^{1, 215}. Mitochondrial CL content was measured by electrospray ionization mass spectrometry¹²⁴. As shown in FIG. 5, we observed that total CL (upper left panel) and the absolute concentration of L4CL in mitochondria (upper right panel) were increased by EPA+DHA. Treatment with EPA+DHA surprisingly and unexpectedly increased the incorporation of DHA into CL, as seen in the 4-fold increase in CL containing three molecules of 18:2n6 and one DHA (“L3 DHA1 CL”) (lower left panel). The net effect was a small decrease in the percent of the CL composed of L4CL (lower right panel). Surprisingly and unexpectedly, there was very little incorporation of EPA into CL with either the standard diet or with supplementation with EPA+DHA (<0.5% of total). There were no differences between SSM and IFM in any parameter. EPA+DHA treatment did not affect State 3 respiration with glutamate, pyruvate or palmitoylcarnitine as substrates.

Based on these results, the invention (at least in part) predicated on increasing the content of CL and L4CL in cardiac mitochondria by supplementation with EPA and/or DHA will improve outcome in HF by preventing MPTP and increasing supercomplex assembly, improving ETC flux and oxidative phosphorylation, decreasing apoptosis and improving LV function and survival. As discussed in our recent paper, HF decreases assembly of mitochondrial supercomplexes comprised of complex I/complex III dimer/complex IV, which may be responsible for the decrease in oxidative phosphorylation⁶. Previous work by others show that elevated levels of CL increase formation of respiratory supercomplexes^{20, 21}. Thus we expect that EPA+DHA supplementation will increase CL content with HF, and prevent the dramatic HF-induced decrease in complex I/complex III dimer/complex IV supercomplex, and improve mitochondrial respiration and LV function. In particular our results indicate that supplementation with DHA alone will be more effective the EPA+DHA.

### Example 3

### Delayed Ca²⁺-Induced MPTP Opening with EPA+DHA Supplementation

Formation of MPTP triggers cardiomyocyte apoptosis and cell death^{12, 130, 132, 217}. In HF the MPTP forms more readily both in the unstressed State 4 and in response to standard stresses, such as a progressive increase in extramitochondrial Ca²⁺¹³. CL is critical for preventing apoptosis in cardiomyocytes; this effect is partially mediated through the anchoring of cytochrome C to the inner mitochondrial membrane by CL¹⁵⁻¹⁹. Studies on the effects of supplementation with EPA+DHA on MPTP formation in isolated cardiac mitochondria were performed in normal male rats fed chow supplemented with EPA+DHA (2.3% of energy intake as EPA+DHA)(n=6/group) for 12 weeks. Two populations of cardiac mitochondria (subsarcolemmal (SSM) and intrafibrillar (IFM)) were isolated, and MPTP formation was assessed using previously published methods^{1, 215, 218}. Briefly, this assay is based on the ability of the mitochondria to take up Ca²⁺, resist swelling and maintain membrane potential^{135, 218-220}. Isolated mitochondria (0.75 mg of protein) were suspended in 2 ml of respiration buffer with 10 mM glutamate, and 5 mM malate at 37° C. in a water-jacketed cuvette. A 5 mM Ca²⁺ solution was continuously infused and free extramitochondrial Ca²⁺ was monitored with Fura-6-F, with the fluorophor calibrate at the end of each experiment.

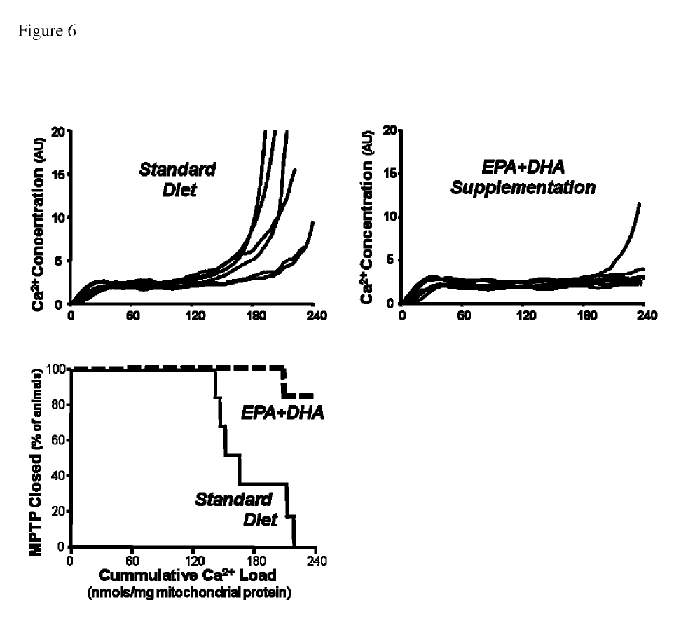

As shown in the upper panel of FIG. 6, there was a sharp increase in extramitochondrial Ca²⁺ as a function of the cumulative amount of infused Ca²⁺ in the mitochondria from all the rats fed the standard diet, which reflects MPTP opening²¹⁸. This effect was not observed in 5 of the 6 rats treated EPA+DHA over the duration of Ca²⁺ infusion employed (FIG. 6). Thus treatment with EPA+DHA delays MPTP formation.

The large increase in total CL and L3,DHA CL was associated with prevention of MPTP formation, demonstrating that they are responsible for the effect presented in FIG. 6. In addition, the lack of major incorporation of EPA into CL demonstrates that DHA supplementation exerts protective effects on MPTP, but EPA does not. In addition, since CL is needed for formation of respiratory supercomplexes comprised of complex I, III & IV and optimal ETC flux, DHA exerts beneficial effect through the formation of supercomplexes and increases in respiration and/or respiration efficiency in mitochondria^{20-23, 221}.

### Example 4

### Dietary Supplementation with DHA Alters Cardiac Mitochondrial Phospholipid Fatty Acid Composition and Prevents Permeability Transition

Treatment with the omega-3 polyunsaturated fatty acids (PUFAs) docosahexaenoic acid (DHA) and eicosapentanoic acid (EPA) exerts cardioprotective effects, and suppresses Ca²⁺-induced opening of the mitochondrial permeability transition pore (MPTP). These effects are associated with increased DHA and EPA, and lower arachidonic acid (ARA) in cardiac phospholipids. While clinical studies suggest the triglyceride lowering effects of DHA and EPA are equivalent, little is known about the independent effects of DHA and EPA on mitochondria function. The effects of dietary supplementation with the omega-3 PUFAs DHA and EPA were compared on cardiac mitochondrial phospholipid fatty acid composition and Ca²⁺-induced MPTP opening.

Experimental Design: The animal protocol was conducted according to the Guideline for the Care and Use of Laboratory Animals (NIH publication 85-23) and was approved by the University of Maryland School of Medicine Institutional Animal Care and Use Committee. Investigators were blinded to treatment when measurements were performed. The animals were maintained on a reverse 12-h light-dark cycle and all procedures were performed in the fed state between 3 and 6 h from the start of the dark phase. Two series of experiments were performed. In the initial studies (Series 1) male Wistar rats weighing 190-200 g were fed a standard low fat diet (CTRL) or modified standard diet containing DHA or EPA at 2.5% of total caloric intake, which corresponds to a human intake of approximately 5.5 g/day (calculated assuming an energy intake of 2000 kcal/day and 9 kcal/g of fat). The rats were maintained on the diet for 8 weeks. Following dietary treatment, rats were anesthetized with isoflurane, blood was drawn, and the heart was harvested for biochemical analysis and mitochondrial isolation. Plasma from these animals was analyzed for free fatty acids and triglyceride levels, and cardiac mitochondria for respiration, Ca²⁺ retention capacity (an index of MPTP opening), VDAC and cyclophilin D, and membrane phospholipid composition as described below. Following completion of Series 1, a second series of animal studies (Series 2) were performed to assess mitochondrial swelling using a light scattering assay, and the effects of different respiratory substrates on Ca²⁺ retention capacity in mitochondria from control and DHA supplemented rats. Animals were treated for 10 weeks and were fed either CTRL or DHA (2.5% of total caloric intake).

Diets: All diets were custom-manufactured (Research Diets Inc., New Brunswick, N.J.), and had 68% of total energy from carbohydrate (38% of total energy from cornstarch, 5% from maltodextrin and 25% from sucrose), 20% protein (casein supplemented with 1-cystine) and 12% energy from fat. In Series 1 the CTRL diet the fat was made up of 35.3% cocoa butter, 39.8% lard, 16.6% soybean oil and 8.3% palm kernel oil (see Table 1 for fatty acid composition).

TABLE 1
Fatty acid compositions of the rodent diets expressed as the molar
percent of total fatty acids in the diet. In Series 1 all diets had 12%
of total energy from fat, 20% from protein and 68% from carbohydrate,
and in Series 2 all diets had 14% of total energy from fat, 20% from
protein and 66% from carbohydrate.

| | |
|---|---|
| Series 1 | Series 2 |

| Fatty Acid | CTRL | DHA | EPA | CRTL | DHA |
|---|---|---|---|---|---|
| C12:0 | 3.9 | 2.2 | 3.9 | 3.4 | 1.5 |
| C14:0 | 1.7 | 4.9 | 1.3 | 1.1 | 3.6 |
| C16:0 | 21.6 | 14.7 | 13.1 | 21.7 | 14.6 |
| C16:1 | 1.7 | 1.8 | — | 0.2 | 1.4 |
| C18:0 | 18.9 | 13.8 | 14.0 | 25.8 | 14.2 |
| C18:1n-9 | 34.9 | 29.7 | 31.6 | 30.3 | 28.8 |
| C18:2n-6 | 13.9 | 11.1 | 13.0 | 13.9 | 14.7 |
| C18:3n-3 | 1.8 | 1.3 | 1.3 | 2.2 | 2.2 |
| C20:5n-3 | — | — | 19.1 | — | — |
| C22:6n-3 | — | 19.1 | — | — | 17.8 |

The DHA diet contained 5.75% of total energy from algal oil that was comprised of 45.6% DHA by mass (DHASCO, Martek Inc, Columbia, Md., USA), with the balance from cocoa butter and soybean oil. The EPA diet had 2.6% of energy from purified fish oil comprised of 95.5% EPA by mass (KD Pharma, Bexbach, Germany), with the balance from cocoa butter, soybean oil, safflower oil and palm kernel oil. DHA and EPA oils contained ascorbyl palmitate (250 ppm) and tocopherols (250 ppm) to prevent peroxidation, which was less than 0.5 meq/kg at the time of manufacture of the diet. All diets were supplemented with the same amount of vitamins (Vitamin Mix V10001, 10 g/kg), minerals (Mineral Mix S10026, 10 g/kg), cellulose (50 g/kg) and choline (2 g/kg). In Series 2, the DHA was again 2.5% of total energy in the diet, but the diets had 66% of total energy from carbohydrate (54% of total energy from cornstarch and 12% from maltodextrin), 20% protein (casein supplemented with 1-cystine) and 14% energy from fat (see Table 1 for fatty acid composition). In the CTRL diet, the fat was made up of 71.5% cocoa butter, 17.1% soybean oil, 7.2% palm oil, 2.8% safflower oil and 1.4% linseed oil. In the DHA diet, 5.75% of algal oil partially replaced cocoa butter. Animals were treated for 8 weeks and mitochondria isolated as in Series 1.

Mitochondrial Preparation: Mitochondria were isolated as previously described²²⁸. LV tissue (400-500 mg) was minced and homogenized in 1:10 cold modified Chappel-Perry buffer (100 mM KCl, 50 mM MOPS, 5 mM MgSO₄, 1 mM ATP, 1 mM EGTA, 2 mg/ml BSA), and the homogenates were centrifuged at 500×g. Subsequent centrifugation allowed for separation and purification of the subsarcolemmal mitochondria. The concentration of mitochondrial protein was measured by the Lowry method using bovine serum albumin as a standard.

Metabolic and Biochemical Parameters: Free fatty acids and triglycerides were assessed in the plasma using commercially available kits (Wako, Richmond, Va.). Mitochondrial proteins were separated by electrophoresis in 4-12% NuPage gels, transferred onto a nitrocellulose membrane, and incubated with specific antibodies to cyclophilin D and voltage-dependent anion channel (VDAC) (1:10,000 and 1:5000, respectively, both from Mitosciences, Eugene, Oreg.). Fluorescence-conjugated secondary antibodies (IRDye 800, 1:10,000; LI-COR Bioscience) were used for incubation before the membranes were scanned with Odyssey® infrared imaging system (LI-COR Bioscience). The digitized image was analyzed with Odyssey® software.

Mitochondrial respiration: Mitochondrial oxygen consumption was measured using a Clark-type oxygen electrode (Qubit Systems, Ontario, Canada). Mitochondria (0.25 mg protein) were suspended in 0.5 ml solution consisting of 100 mM KCl, 50 mM MOPS, 5 mM KH₂PO₄, 1 mM EGTA, and 0.5 mg fatty acid-free bovine serum albumin, at pH 7.4 and 37° C. State 3 (ADP-stimulated) and state 4 (non-phosphorylating) respiration were measured with glutamate+malate (10 and 5 mM, respectively), pyruvate+malate (10 and 5 mM, respectively) and palmitoylcarnitine+malate (40 μM and 5 mM, respectively) to assess respiration through complex I-IV, while succinate+rotenone (10 mM and 7.5 μM, respectively), were used to assess respiration through complex II-IV of the ETC exclusively. State 4 respiration was also measured in the presence of oligomycin to inhibit the mitochondrial ATP synthase.

Ca²⁺ Retention Capacity: The capacity for mitochondrial to retain Ca²⁺, an established index of MPTP opening, was assessed in isolated mitochondria as previously described in detail²²⁸. Briefly, 0.5 mg of mitochondrial protein were suspended in respiration buffer in the absence of bovine serum albumin and the presence of 5 μM EGTA, 1 mM MgCl₂, 10 mM glutamate and 5 mM malate. A 5 mM calcium solution was continuously infused at a rate of 5 μl/min for 20 min, and free Ca²⁺ was monitored by use of 0.7 μl Fura-6-F (0.07 mM) at 37° C. using a fluorescence spectrometer with excitation wavelengths for the free and calcium-bound forms of 340 and 380 nm, respectively, and emission wavelength of 550 nm. Opening of the MPTP was defined as the point where the extramitochondrial [Ca²⁺] reached twice baseline values²²⁶.

For Series 2 a high throughput Ca²⁺ retention assay was developed to allow evaluation of the effects of mitochondrial respiratory substrates on the delay in MPTP induced by DHA supplementation. The assay was modified from Basso et al²²³, and was performed using a 96-well fluorescence plate reader (FLUOstar Optima, BMG Labtech, Germany). Briefly, 25 μg of mitochondria were resuspended in 200 μL of the same buffer used above, but with varying substrates; either glutamate+malate (10 and 5 mM, respectively), pyruvate+malate (10 and 5 mM, respectively), palmitoylcarnitine+malate (40 μM and 5 mM, respectively) or succinate (10 mM) with rotenone (7.5 μM). Extramitochondrial Ca²⁺ was monitored using 1 μM Calcium Green 5N and fluorescence measured at 485 nm and 538 nm for excitation and emission wavelengths respectively. Automated additions of 25 nmoles Ca²⁺/mg mitochondrial protein were performed at regular 7 minute intervals and fluorescence measured every 17 seconds for 160 min at 37° C.

Ca²⁺-Induced Swelling: In Series 2 light scattering, an index of Ca²⁺-induced swelling was monitored using a 96 well spectrophotometric plate reader (SpectraMax, Molecular Devices, USA). Briefly, 25 μg of mitochondria were resuspended in 200 μL the same buffer as used for the Ca²⁺ retention capacity assay. Baseline absorbance at 540 nm was read at 7 second intervals for 2 min, then either 50 or 100 nmoles Ca²⁺ was rapidly added to the wells and the absorbance was read for 15 min at 37° C.

Membrane Lipid Composition: Cardiac phospholipid fatty acid composition was assessed in a subset of animals from Series 1 (n=7-9/group) on isolated cardiac mitochondria homogenates by gas chromatography with a flame ionization detector according to a modification of the transesterification method as previously described²²⁸. CL composition was assessed on isolated cardiac mitochondria by electrospray ionization mass spectrometry using 1,1′,2,2′-tetramyristoyl CL as an internal standard as previously described (n=9/group)^{228,229, 230}.

Statistical Analyses: Mean values are presented ±SEM, and the level of significance was set at p<0.05. Comparisons between groups were made with a one-way analysis of variance (ANOVA) and the Bonferoni post hoc test. Analysis of non-normal data sets was done with Kruskal-Wallis ANOVA on ranks and post hoc comparisons were made using Dunn's method. A two-way repeated measure ANOVA with a Holm-Sidak post hoc test was performed when appropriate.

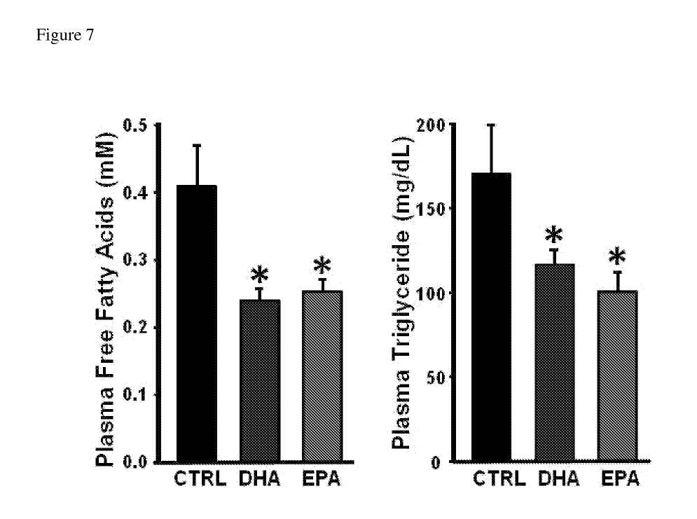

Results: Body and cardiac masses were unaffected by diet (Table 2) and mitochondrial yield not was different among groups. EPA and DHA lowered plasma free fatty acid and triglyceride concentrations to a similar extent compared to CTRL (FIG. 7), as previously shown in humans²²⁴.

TABLE 2
Body mass, organ mass, and mitochondrial yield.

| | Control | DHA | EPA |
|---|---|---|---|
| Terminal Body Mass (g) | 503 ± 15 | 501 ± 19 | 486 ± 29 |
| LV Mass (g) | 0.92 ± 0.04 | 0.94 ± 0.05 | 0.89 ± 0.07 |
| RV Mass (g) | 0.32 ± 0.01 | 0.30 ± 0.02 | 0.29 ± 0.03 |
| Biatrial Mass (g) | 0.09 ± 0.01 | 0.08 ± 0.01 | 0.09 ± 0.02 |
| Liver Mass (g) | 16.0 ± 1.1 | 14.8 ± 0.5 | 15.5 ± 1.5 |
| Mitochondrial Yield | 18.1 ± 3.0 | 18.1 ± 2.2 | 19.0 ± 1.5 |
| (mg mito protein/g wet wt) | | | |

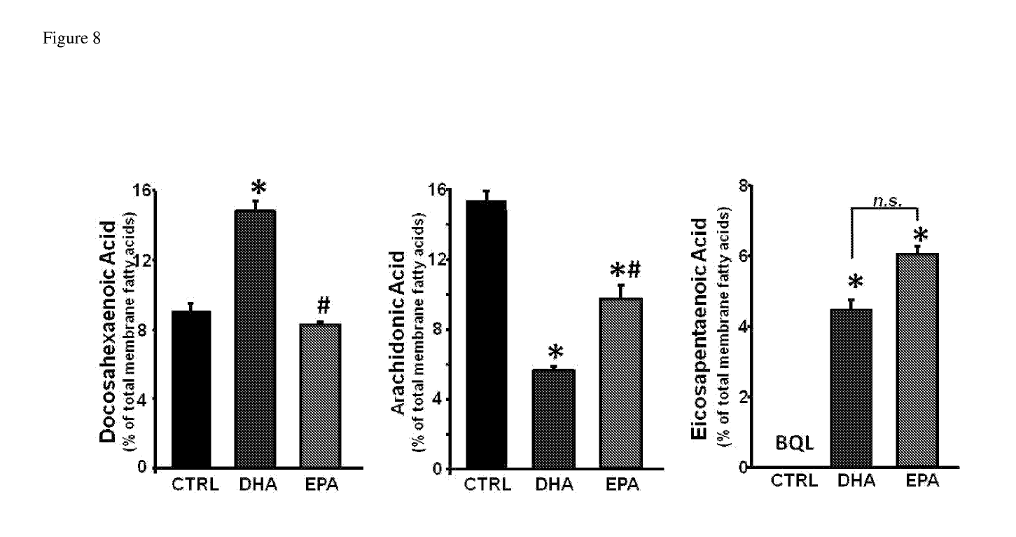

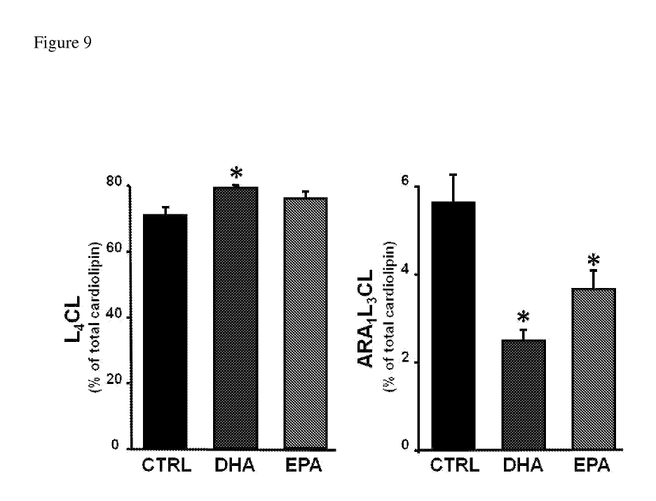

Mitochondrial Phospholipid Composition: EPA was not detected in the CTRL group. The DHA diet significantly increased DHA and EPA, and decreased ARA in mitochondrial phospholipids. On the other hand, the EPA diet did not affect DHA levels, and only modestly decreased ARA levels, and increased EPA in a manner similar to treatment with DHA (FIG. 8). Dihomogammalinolenic Acid (20:3n6), an intermediate in the synthesis of ARA from linoleic acid, was not detected in the CTRL group, but was increased to a similar extent by supplementation with either DHA or EPA (p<0.05) (Table 3).

TABLE 3
Mitochondrial phospholipid fatty acid composition expressed as molar
percent of total phospholipid fatty acid.

| Fatty Acid | Control | DHA | EPA |
|---|---|---|---|
| C16:0 | 12.2 ± 0.8 | 13.3 ± 0.5 | 10.8 ± 0.6 |
| C16:1 | BQL | BQL | 4.5 ± 1.8 |
| C18:0 | 20.0 ± 0.4 | 18.9 ± 0.5 | 19.8 ± 0.8 |
| C18:1n9 | 14.3 ± 0.8 | 13.0 ± 1.0 | 11.8 ± 1.0 |
| C18:1n7 | 4.5 ± 0.2 | 3.7 ± 0.2* | 4.2 ± 0.2 |
| C18:2n6 | 22.0 ± 0.9 | 23.1 ± 1.0 | 22.4 ± 0.8 |
| C20:3n6 | BQL | 1.6 ± 0.2* | 1.1 ± 0.2* |
| C20:4n6 | 14.6 ± 1.0 | 5.7 ± 0.2* | 9.8 ± 0.8*# |
| C20:5n3 | BQL | 4.4 ± 0.3* | 6.0 ± 0.2* |
| C22:6n3 | 9.1 ± 0.5 | 14.8 ± 0.6* | 8.3 ± 0.2# |
| Total ω-3 PUFA | 9.1 ± 0.5 | 19.2 ± 0.5* | 14.3 ± 0.3*# |

Data are expressed as percent of total mitochondrial membrane phospholipid content.
BQL, below the quantifiable limit (limit of detection = 0.41% of total phospholipid fatty acids).
Data are the mean ± SEM.
n = 7-8/group.
*p < 0.05 compared to the control group;
#p < 0.001 compared to DHA

The composition of CL was altered by DHA, showing an increase in L₄CL (FIG. 9), the major and most critical species of CL²³¹. In addition, there was a strong trend to increase total CL content in the DHA group (13.2±0.8 nmols/mg mito prot) compared to CTRL (10.7±0.8) (p<0.08), with no effect in the EPA treated animals (11.1±0.8). There was a decrease in CL species containing one ARA and three linoleic acid moieties (ARA₁L₃CL) in both DHA and EPA groups compared to CTRL (FIG. 9).

Mitochondrial Respiration: State 3 respiration with glutamate+malate, pyruvate+malate, palmitoylcarnitine+malate, or succinate+rotenone as substrates was unaffected by dietary treatment. DHA treatment decreased state 4 respiration by 30% and the increased RCR by 70% with pyruvate+malate as the substrate in both the absence and presence of oligomycin to eliminate any ATP turnover (p<0.05) (Table 4); treatment with EPA had no effect. Neither state 4 respiration or the respiratory control ratio (RCR) with glutamate+malate, palmitoylcarnitine+malate, or succinate+rotenone as substrates were affected by treatment (Table 4). The P:O ratio (ADP added:Oxygen consumed) was not different among groups with any of the substrates (Table 4), indicating no change in respiratory coupling.

TABLE 4
Mitochondrial Respiration

| | Control | DHA | EPA |
|---|---|---|---|
| Glutamate + Malate | | | |
| State 3 | 120.2 ± 10.0 | 115.2 ± 16.2 | 119.3 ± 13.4 |
| State 4 (− oligomycin) | 34.9 ± 2.1 | 35.3 ± 2.9 | 32.1 ± 4.8 |
| State 4 (+ oligomycin) | 20.4 ± 1.8 | 17.2 ± 2.0 | 19.1 ± 3.3 |
| RCR | 7.2 ± 1.1 | 7.0 ± 0.9 | 7.0 ± 1.0 |
| P:O | 2.57 ± 0.20 | 2.37 ± 0.18 | 2.76 ± 0.23 |
| Pyruvate + Malate | | | |
| State 3 | 226.6 ± 19.5 | 240.4 ± 28.7 | 221.2 ± 31.0 |
| State 4 (− oligomycin) | 78.1 ± 4.4 | 54.0 ± 3.2* | 63.5 ± 6.4 |
| State 4 (+ oligomycin) | 50.1 ± 4.1 | 34.8 ± 4.2* | 43.3 ± 5.2 |
| RCR | 4.5 ± 0.2 | 7.7 ± 1.0* | 5.1 ± 0.3 |
| P:O | 2.60 ± 0.17 | 2.48 ± 0.09 | 2.44 ± 0.30 |
| Palmityl-carnitine + Malate | | | |
| State 3 | 265.2 ± 28.2 | 265.7 ± 34.4 | 243.7 ± 28.1 |
| State 4 (− oligomycin) | 59.7 ± 4.2 | 42.6 ± 1.9 | 59.4 ± 7.0 |
| State 4 (+ Oligomycin) | 32.0 ± 3.5 | 24.9 ± 2.6 | 29.7 ± 3.8 |
| RCR | 8.8 ± 1.0 | 11.4 ± 1.8 | 9.5 ± 1.8 |
| P:O | 2.46 ± 0.13 | 2.50 ± 0.12 | 2.57 ± 0.14 |
| Succinate + Rotenone | | | |
| State 3 | 316.8 ± 31.1 | 325.2 ± 19.6 | 307.2 ± 37.1 |
| State 4 (− oligomycin) | 105.7 ± 9.7 | 99.3 ± 7.0 | 102.1 ± 11.5 |
| State 4 (+ oligomycin) | 82.5 ± 9.5 | 84.7 ± 10.1 | 80.3 ± 11.7 |
| RCR | 4.1 ± 0.4 | 4.1 ± 0.4 | 4.1 ± 0.5 |
| P:O | 1.57 ± 0.09 | 1.48 ± 0.06 | 1.48 ± 0.08 |

Data are the mean ± SEM. n = 7 or 8/group.
All Rates are expressed in ng atoms O · mg⁻¹ · min⁻¹.
*p < 0.05 compared to the control group.
The RCR, defined as the ratio of State 3 to State 4 respiration rate, was calculated from the State 4 rate with oligomycin.
The P:O ratio was calculated from measurements made without oligomycin.

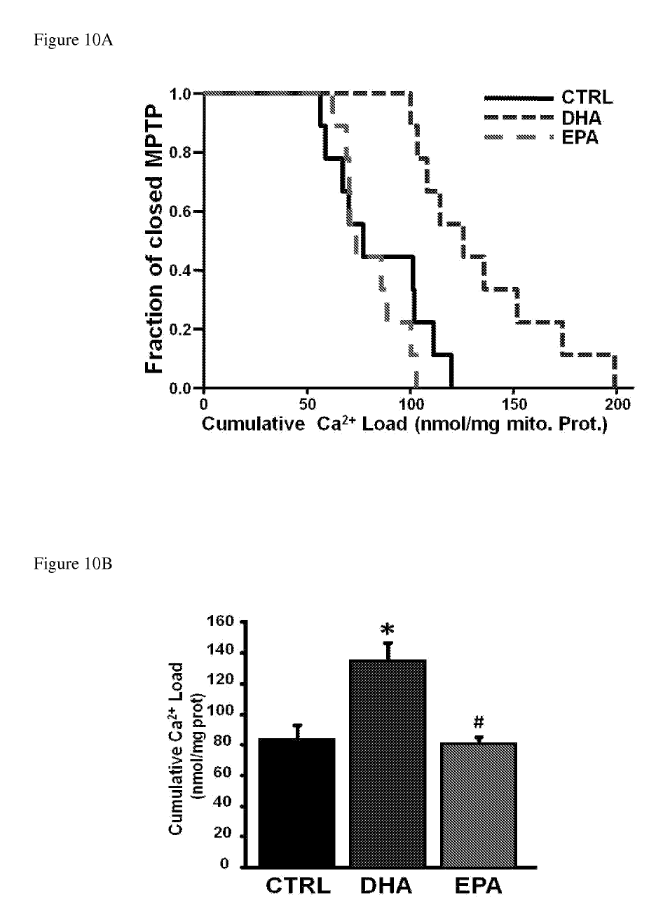

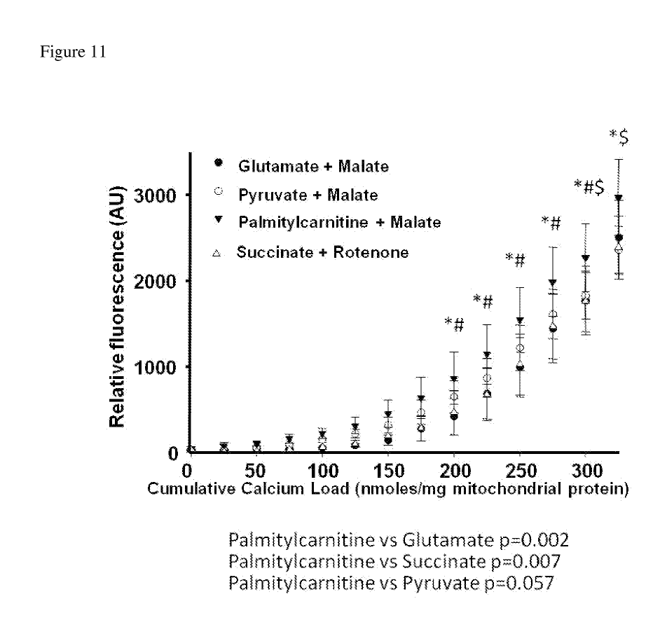

Ca²⁺ Retention Capacity: Compared to the CTRL and EPA treated groups, DHA significantly increased the Ca²⁺ retention capacity, an index of MPTP opening (FIG. 10). As expected, addition of 100 nM CsA lead to a significant increase in Ca²⁺ required to elicit MPTP in the CTRL group (83.0±8.7 nmol Ca²⁺/mg mito prot vs 144.8±20.0, p<0.05) and a similar effect in the EPA group (80.8±4.9 vs 130.6±12.8, p<0.05). There was no difference in the DHA group with the addition of CsA (134.9±11.4 vs 147.0±17.3, NS). Cyclophilin-D is a key regulatory component of the MPTP²²², however western blot analysis found no effect of any diet on cyclophilin-D protein expression (Table 5). The voltage-dependent anion channel (VDAC) has been proposed to play a role in regulation of the MPTP, however protein expression of VDAC1 and VDAC2 was similar among groups (Table 5).

TABLE 5
Western blot results for VDAC1, VDAC2 and cyclophilin D in isolated
mitochondria as assessed by densitometry. There were no differences
among groups.

| | CTRL | DHA | EPA |
|---|---|---|---|
| VDAC 1 | 1.00 ± 0.13 | 1.03 ± 0.22 | 0.87 ± 0.16 |
| VDAC 2 | 1.00 ± 0.15 | 1.02 ± 0.20 | 0.94 ± 0.17 |
| Cyclophilin D | 1.00 ± 0.08 | 1.01 ± 0.10 | 1.00 ± 0.08 |

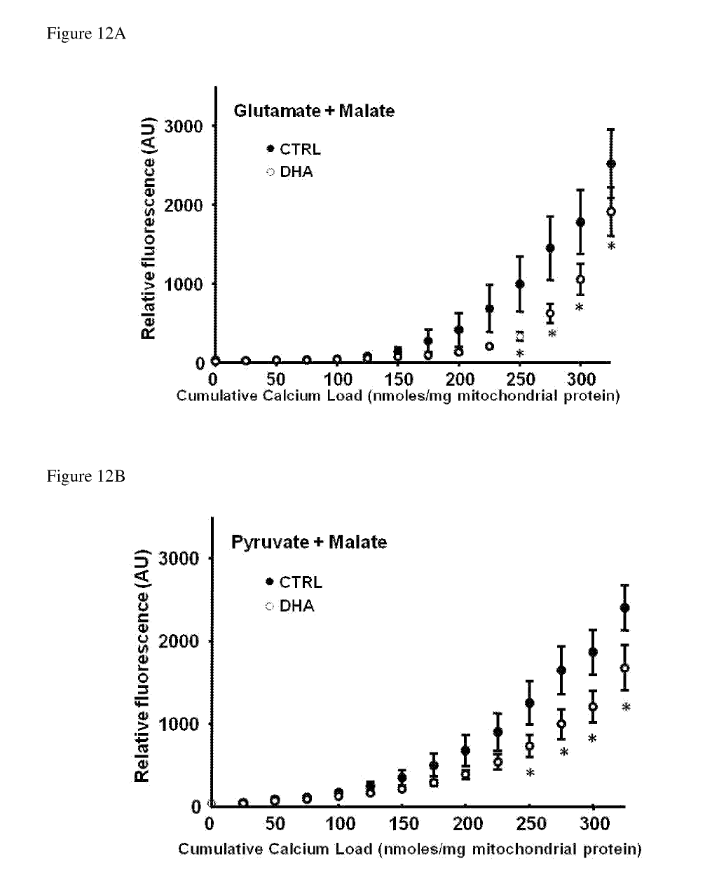

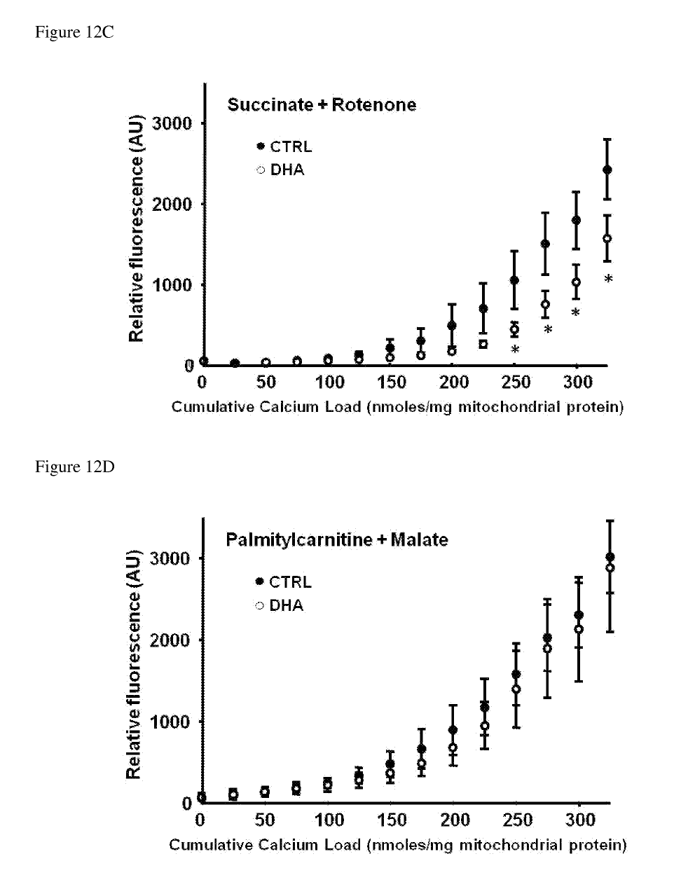

In Series 2, a high throughput assay was used to compare Ca²⁺ retention capacity of mitochondria from DHA supplemented hearts to CTRL, in the presence of different respiratory substrates. First, in the control diet, there was a decreased Ca²⁺ retention capacity with palmitoylcarnitine+malate when compared to glutamate+malate or succinate+rotenone (p<0.007; FIG. 11), and a strong trend when compared to pyruvate+malate (p=0.057). Mitochondria from rats supplemented with DHA had significantly enhanced Ca²⁺ retention capacity compared to CTRL animals, as reflected in lower extramitochondrial Ca²⁺ for a given cumulative Ca²⁺ load with all substrates except palmitoylcarnitine+malate (FIG. 12).

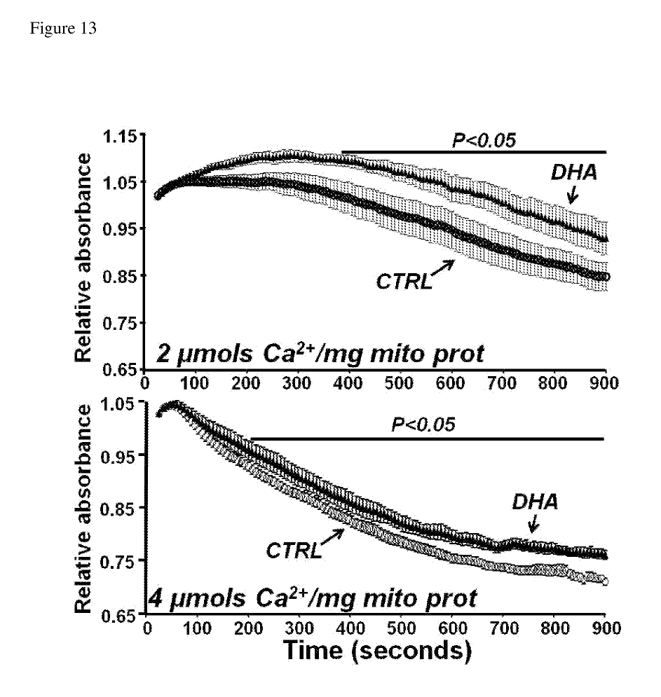

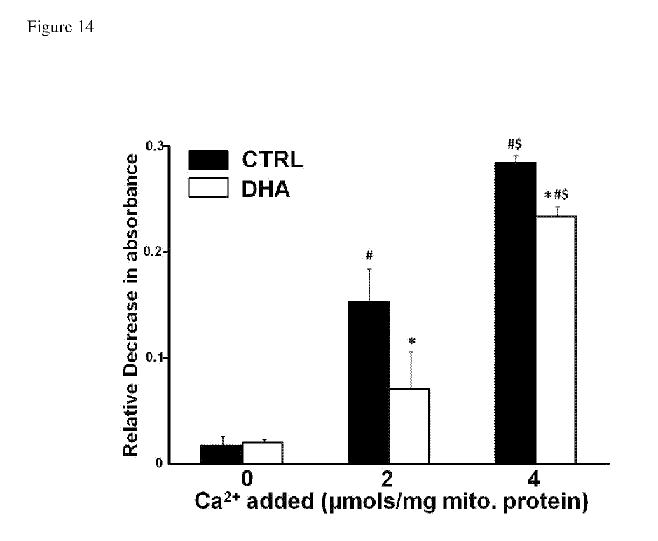

Mitochondrial Swelling: In the mitochondria from CTRL rats there was a dose-dependent decrease in absorbance at 540 nm with the addition of Ca²⁺, which was significantly attenuated with DHA supplementation (FIGS. 13 and 14).

The result support the following conclusion: 1) DHA supplementation delayed MPTP opening in response to Ca²⁺ compared to animals fed the standard diet or supplemented with EPA, and 2) this effect is associated with a greater increase in total omega-3 PUFA in cardiac mitochondrial phospholipids with DHA supplementation compared to EPA, which corresponds with a greater reduction in the amount of ARA and an increase in L₄CL. These differences between DHA and EPA occurred despite equivalent triglyceride lowering effects in the present investigation and in clinical studies^{224, 227}, suggesting that the lipid lowering effects of DHA and EPA are independent of phospholipid remodeling, as previously proposed²²⁵. Thus the results show a novel and important difference between DHA and EPA supplementation: DHA causes more extensive alterations in mitochondrial phospholipid fatty acid composition and delays Ca²⁺-induced MPTP opening, despite lipid lowering effects that are similar to EPA. Since mitochondrial dysfunction and MPTP opening in cardiac mitochondria appear to play an important role in the development and progression of HF, these finding suggest the treatment with DHA alone would be an effective treatment for HF patients, and would be superior to treatment with EPA or a combination of EPA+DHA.

While the invention has been described with reference to certain particular embodiments thereof, those skilled in the art will appreciate that various modifications may be made without departing from the spirit and scope of the invention. The scope of the appended claims is not to be limited to the specific embodiments described.

### REFERENCES

All patents and publications mentioned in this specification are indicative of the level of skill of those skilled in the art to which the invention pertains. Each cited patent and publication is incorporated herein by reference in its entirety. All of the following references have been cited in this application:
(1) Rosca M G, Vazquez E C, Kerner J, Parland W, Chandler M P, Stanley W C, Sabbah H N, Hoppel C L. Cardiac mitochondria in coronary microembolization-induced heart failure: decrease in respirasomes and oxidative phosphorylation. Cardiovasc Res 2008; 80:30-9.
(2) Duda M K, O'shea K M, Tintinu A, Xu W, Khairallah R J, Barrows B R, Chess D J, Azimzadeh A M, Harris W S, Sharov V G, Sabbah H N, Stanley W C. Fish oil, but not flaxseed oil, decreases inflammation and prevents pressure overload-induced cardiac dysfunction. Cardiovasc Res 2009 February 1; 81(2):319-27.
(3) Mozaffarian D, Bryson C L, Lemaitre R N, Burke G L, Siscovick D S. Fish intake and risk of incident heart failure. J Am Coll Cardiol 2005 June 21; 45(12):2015-21.
(4) Sharov V G, Todor A, Khanal S, Imai M, Sabbah H N. Cyclosporine A attenuates mitochondrial permeability transition and improves mitochondrial respiratory function in cardiomyocytes isolated from dogs with heart failure. J Mol Cell Cardiol 2007 January; 42(1):150-8.
(5) Pepe S, McLennan P L. Cardiac membrane fatty acid composition modulates myocardial oxygen consumption and postischemic recovery of contractile function. Circulation 2002 May 14; 105(19):2303-8.
(6) Fiaccavento R, Carotenuto F, Minieri M, Masuelli L, Vecchini A, Bei R, Modesti A, Binaglia L, Fusco A, Bertoli A, Forte G, Carosella L, Di N P. Alpha-linolenic acid-enriched diet prevents myocardial damage and expands longevity in cardiomyopathic hamsters. Am J Pathol 2006 December; 169(6):1913-24.
(7) Kaye D M, Krum H. Drug discovery for heart failure: a new era or the end of the pipeline? Nat Rev Drug Discov 2007 February; 6(2):127-39.
(8) Mann D L, Bristow M R. Mechanisms and models in heart failure: the biomechanical model and beyond. Circulation 2005 May 31; 111(21):2837-49.
(9) Gissi-Hf I. Effect of n-3 polyunsaturated fatty acids in patients with chronic heart failure (the GISSI-HF trial): a randomised, double-blind, placebo-controlled trial. Lancet 2008 January 4; 372:1223-30.
(10) Neubauer S. The failing heart—an engine out of fuel. N Engl J Med 2007 March 15; 356(11):1140-51.
(11) Ingwall J S, Weiss R G. Is the failing heart energy starved? On using chemical energy to support cardiac function. Circ Res 2004 July 23; 95(2):135-45.
(12) Javadov S, Karmazyn M. Mitochondrial permeability transition pore opening as an endpoint to initiate cell death and as a putative target for cardioprotection. Cell Physiol Biochem 2007; 20(1-4):1-22.
(13) Javadov S, Huang C, Kirshenbaum L, Karmazyn M. NHE-1 inhibition improves impaired mitochondrial permeability transition and respiratory function during postinfarction remodelling in the rat. J Mol Cell Cardiol 2005 January; 38(1):135-43.
(14) Pepe S, McLennan P L. (n-3) Long chain PUFA dose-dependently increase oxygen utilization efficiency and inhibit arrhythmias after saturated fat feeding in rats. J Nutr 2007 November; 137(11):2377-83.
(15) McMillin J B, Dowhan W. Cardiolipin and apoptosis. Biochim Biophys Acta 2002 December 30; 1585(2-3):97-107.
(16) Ostrander D B, Sparagna G C, Amoscato A A, McMillin J B, Dowhan W. Decreased cardiolipin synthesis corresponds with cytochrome c release in palmitate-induced cardiomyocyte apoptosis. J Biol Chem 2001 October 12; 276(41):38061-7.
(17) Robinson N C. Functional binding of cardiolipin to cytochrome c oxidase. J Bioenerg Biomembr 1993 April; 25(2):153-63.
(18) Petrosillo G, Casanova G, Matera M, Ruggiero F M, Paradies G. Interaction of peroxidized cardiolipin with rat-heart mitochondrial membranes: induction of permeability transition and cytochrome c release. FEBS Lett 2006 November 27; 580(27):6311-6.
(19) Ott M, Zhivotovsky B, Orrenius S. Role of cardiolipin in cytochrome c release from mitochondria. Cell Death Differ 2007 July; 14(7):1243-7.
(20) Zhang M, Mileykovskaya E, Dowhan W. Cardiolipin is essential for organization of complexes III and IV into a supercomplex in intact yeast mitochondria. J Biol Chem 2005 August 19; 280(33):29403-8.
(21) Zhang M, Mileykovskaya E, Dowhan W. Gluing the respiratory chain together. Cardiolipin is required for supercomplex formation in the inner mitochondrial membrane. J Biol Chem 2002 November 15; 277(46):43553-6.
(22) Fry M, Green D E. Cardiolipin requirement by cytochrome oxidase and the catalytic role of phospholipid. Biochem Biophys Res Commun 1980 April 29; 93(4):1238-46.
(23) Fry M, Green D E. Cardiolipin requirement for electron transfer in complex I and III of the mitochondrial respiratory chain. J Biol Chem 1981 February 25; 256(4):1874-80.
(24) Vemuri M, Kelley D S, Mackey B E, Rasooly R, Bartolini G. Docosahexaenoic Acid (DHA) But Not Eicosapentaenoic Acid (EPA) Prevents Trans-10, Cis-12 Conjugated Linoleic Acid (CLA)-Induced Insulin Resistance in Mice. Metab Syndr Relat Disord 2007 December; 5(4):315-22.
(25) Cazzola R, Russo-Volpe S, Miles E A, Rees D, Banerjee T, Roynette C E, Wells S J, Goua M, Wahle K W, Calder P C, Cestaro B. Age- and dose-dependent effects of an eicosapentaenoic acid-rich oil on cardiovascular risk factors in healthy male subjects. Atherosclerosis 2007 July; 193(1): 159-67.
(26) Thies F, Nebe-von-Caron G, Powell J R, Yaqoob P, Newsholme E A, Calder P C. Dietary supplementation with eicosapentaenoic acid, but not with other long-chain n3 or n-6 polyunsaturated fatty acids, decreases natural killer cell activity in healthy subjects aged >55 y. Am J Clin Nutr 2001 March; 73(3):539-48.
(27) Rees D, Miles E A, Banerjee T, Wells S J, Roynette C E, Wahle K W, Calder P C. Dose-related effects of eicosapentaenoic acid on innate immune function in healthy humans: a comparison of young and older men. Am J Clin Nutr 2006 February; 83(2):331-42.
(28) Calder P C. n-3 polyunsaturated fatty acids, inflammation, and inflammatory diseases. Am J Clin Nutr 2006 June; 83(6 Suppl):1505S-19S.
(29) Calder P C. The relationship between the fatty acid composition of immune cells and their function. Prostaglandins Leukot Essent Fatty Acids 2008 September; 79(3-5):101-8.
(30) Hunt S A, Baker D W, Chin M H, Cinquegrani M P, Feldmanmd A M, Francis G S, Ganiats T G, Goldstein S, Gregoratos G, Jessup M L, Noble R J, Packer M, Silver M A, Stevenson L W, Gibbons R J, Antman E M, Alpert J S, Faxon D P, Fuster V, Gregoratos G, Jacobs A K, Hiratzka L F, Russell R O, Smith S C, Jr. ACC/AHA Guidelines for the Evaluation and Management of Chronic Heart Failure in the Adult: Executive Summary A Report of the American College of Cardiology/American Heart Association Task Force on Practice Guidelines (Committee to Revise the 1995 Guidelines for the Evaluation and Management of Heart Failure): Developed in Collaboration With the International Society for Heart and Lung Transplantation; Endorsed by the Heart Failure Society of America. Circulation 2001 December 11; 104(24):2996-3007.
(31) Katz A M. Heart failure: pathophysiology, molecular biology, and clinical management. Philadelphia: Lippincott Williams & Wilkins; 2000.
(32) Gradman A H, Alfayoumi F. From left ventricular hypertrophy to congestive heart failure: management of hypertensive heart disease. Prog Cardiovasc Dis 2006 March; 48(5):326-41.
(33) Bhatia R S, Tu J V, Lee D S, Austin P C, Fang J, Haouzi A, Gong Y, Liu P P. Outcome of heart failure with preserved ejection fraction in a population-based study. N Engl J Med 2006 July 20; 355(3):260-9.
(34) Owan T E, Hodge D O, Herges R M, Jacobsen S J, Roger V L, Redfield MM. Trends in prevalence and outcome of heart failure with preserved ejection fraction. N Engl J Med 2006 July 20; 355(3):251-9.
(35) Coats A J. Angiotensin type-1 receptor blockers in heart failure. Prog Cardiovasc Dis 2002 January; 44(4):231-42.
(36) Bristow M R, Gilbert E M, Abraham W T, Adams K F, Fowler M B, Hershberger R E, Kubo S H, Narahara K A, Ingersoll H, Krueger S, Young S, Shusterman N. Carvedilol produces dose-related improvements in left ventricular function and survival in subjects with chronic heart failure. MOCHA Investigators. Circulation 1996 December 1; 94(11):2807-16.
(37) Colucci W S, Packer M, Bristow M R, Gilbert E M, Cohn J N, Fowler M B, Krueger S K, Hershberger R, Uretsky B F, Bowers J A, Sackner-Bernstein J D, Young S T, Holcslaw T L, Lukas M A. Carvedilol inhibits clinical progression in patients with mild symptoms of heart failure. US Carvedilol Heart Failure Study Group. Circulation 1996 December 1; 94(11):2800-6.
(38) Sabbah H N, Stanley W C, Sharov V G, Mishima T, Tanimura M, Benedict C R, Hegde S, Goldstein S. Effects of dopamine beta-hydroxylase inhibition with nepicastat on the progression of left ventricular dysfunction and remodeling in dogs with chronic heart failure. Circulation 2000 October 17; 102(16):1990-5.
(39) Cohn J N, Pfeffer M A, Rouleau J, Sharpe N, Swedberg K, Straub M, Wiltse C, Wright T J. Adverse mortality effect of central sympathetic inhibition with sustained-release moxonidine in patients with heart failure (MOXCON). Eur J Heart Fail 2003 October; 5(5):659-67.
(40) Swedberg K, Bristow M R, Cohn J N, Dargie H, Straub M, Wiltse C, Wright T J. Effects of sustained-release moxonidine, an imidazoline agonist, on plasma norepinephrine in patients with chronic heart failure. Circulation 2002 April 16; 105(15):1797-803.
(41) Bristow M. Etomoxir: a new approach to treatment of chronic heart failure. Lancet 2000 November 11; 356(9242):1621-2.
(42) From A H. Should manipulation of myocardial substrate utilization patterns be a component of the congestive heart failure therapeutic paradigm? J Card Fail 1998 June; 4(2):127-9.
(43) Sabbah H H, Stanley W C. Partial fatty acid oxidation inhibitors: a potentially new class of drugs for heart failure. Eur J Heart Fail 2002 January; 4(1):3-6.
(44) Tang W H, Francis G S. Novel pharmacological treatments for heart failure. Expert Opin Investig Drugs 2003 November; 12(11): 1791-801.
(45) Napoli C, Stanley W C, Ignarro L J. Nutrition and cardiovascular disease: Putting a pathogenic framework into focus. Cardiovasc Res 2007 January 15; 73(2):253-6.
(46) Stanley W C, Hoppel C L. Mitochondrial dysfunction in heart failure: potential for therapeutic interventions? Cardiovasc Res 2000 March; 45(4):805-6.
(47) Harris W S. Alpha-linolenic acid: a gift from the land? Circulation 2005 June 7; 111(22):2872-4.
(48) Harris W S, Mozaffarian D, Rimm E, Kris-Etherton P, Rudel L L, Appel L J, Engler M M, Engler M B, Sacks F. Omega-6 Fatty Acids and Risk for Cardiovascular Disease. A Science Advisory From the American Heart Association Nutrition Subcommittee of the Council on Nutrition, Physical Activity, and Metabolism; Council on Cardiovascular Nursing; and Council on Epidemiology and Prevention. Circulation 2009 Jan. 26.
(49) Schacky C, Harris W S. Cardiovascular benefits of omega-3 fatty acids. Cardiovasc Res 2007 January 15; 73(2):310-5.
(50) Lichtenstein A H, Appel L J, Brands M, Carnethon M, Daniels S, Franch H A, Franklin B, Kris-Etherton P, Harris W S, Howard B, Karanja N, Lefevre M, Rudel L, Sacks F, Van Horn L, Winston M, Wylie-Rosett J. Diet and lifestyle recommendations revision 2006: a scientific statement from the American Heart Association Nutrition Committee. Circulation 2006 July 4; 114(1):82-96.
(51) Kris-Etherton P M, Harris W S, Appel L J. Fish consumption, fish oil, omega-3 fatty acids, and cardiovascular disease. Circulation 2002 November 19; 106(21):2747-57.
(52) Albert C M, Oh K, Whang W, Manson J E, Chae C U, Stampfer M J, Willett W C, Hu F B. Dietary alpha-linolenic acid intake and risk of sudden cardiac death and coronary heart disease. Circulation 2005 November 22; 112(21):3232-8.
(53) Mozaffarian D. Does alpha-linolenic acid intake reduce the risk of coronary heart disease? A review of the evidence. Altern Ther Health Med 2005 May; 11(3):24-30.
(54) Wang C, Harris W S, Chung M, Lichtenstein A H, Balk E M, Kupelnick B, Jordan H S, Lau J. n-3 Fatty acids from fish or fish-oil supplements, but not alpha-linolenic acid, benefit cardiovascular disease outcomes in primary- and secondary-prevention studies: a systematic review. Am J Clin Nutr 2006 July; 84(1):5-17.
(55) Harris W S, Sands S A, Windsor S L, Ali H A, Stevens T L, Magalski A, Porter C B, Borkon A M. Omega-3 fatty acids in cardiac biopsies from heart transplantation patients: correlation with erythrocytes and response to supplementation. Circulation 2004 September 21; 110(12):1645-9.
(56) Metcalf R G, James M J, Gibson R A, Edwards J R, Stubberfield J, Stuklis R, Roberts-Thomson K, Young G D, Cleland L G. Effects of fish-oil supplementation on myocardial fatty acids in humans. Am J Clin Nutr 2007 May; 85(5):1222-8.
(57) Gebauer S K, Psota T L, Harris W S, Kris-Etherton P M. n-3 fatty acid dietary recommendations and food sources to achieve essentiality and cardiovascular benefits. Am J Clin Nutr 2006 June; 83(6 Suppl):1526S-35S.
(58) Harris W S. n-3 fatty acids and serum lipoproteins: human studies. Am J Clin Nutr 1997 May; 65(5 Suppl):1645S-54S.
(59) Lichtenstein A H, Appel L J, Brands M, Carnethon M, Daniels S, Franch H A, Franklin B, Kris-Etherton P, Harris W S, Howard B, Karanja N, Lefevre M, Rudel L, Sacks F, Van Horn L, Winston M, Wylie-Rosett J. Summary of American Heart Association Diet and Lifestyle Recommendations revision 2006. Arterioscler Thromb Vasc Biol 2006 October; 26(10):2186-91.
(60) Weber P, Raederstorff D. Triglyceride-lowering effect of omega-3 LC-polyunsaturated fatty acids—a review. Nutr Metab Cardiovasc Dis 2000 February; 10(1):28-37.
(61) Goodfellow J, Bellamy M F, Ramsey M W, Jones C J, Lewis M J. Dietary supplementation with marine omega-3 fatty acids improve systemic large artery endothelial function in subjects with hypercholesterolemia. J Am Coll Cardiol 2000 February; 35(2):265-70.
(62) Leaf A, Kang J X, Xiao Y F, Billman G E. Clinical prevention of sudden cardiac death by n-3 polyunsaturated fatty acids and mechanism of prevention of arrhythmias by n-3 fish oils. Circulation 2003 June 3; 107(21):2646-52.
(63) Leaf A, Xiao Y F, Kang J X, Billman G E. Prevention of sudden cardiac death by n-3 polyunsaturated fatty acids. Pharmacol Ther 2003 June; 98(3):355-77.
(64) Das U N. Beneficial effect(s) of n-3 fatty acids in cardiovascular diseases: but, why and how? Prostaglandins Leukot Essent Fatty Acids 2000 December; 63(6):351-62.
(65) Khan F, Elherik K, Bolton-Smith C, Barr R, Hill A, Murrie I, Belch J J. The effects of dietary fatty acid supplementation on endothelial function and vascular tone in healthy subjects. Cardiovasc Res 2003 October 1; 59(4):955-62.
(66) Lopez-Garcia E, Schulze M B, Manson J E, Meigs J B, Albert C M, Rifai N, Willett W C, Hu F B. Consumption of (n-3) fatty acids is related to plasma biomarkers of inflammation and endothelial activation in women. J Nutr 2004 July; 134(7): 1806-11.
(67) Sethi S, Ziouzenkova O, Ni H, Wagner D D, Plutzky J, Mayadas T N. Oxidized omega-3 fatty acids in fish oil inhibit leukocyte-endothelial interactions through activation of PPAR alpha. Blood 2002 August 15; 100(4):1340-6.
(68) Treasure C B, Vita J A, Cox D A, Fish R D, Gordon J B, Mudge G H, Colucci W S, Sutton M G, Selwyn A P, Alexander R W. Endothelium-dependent dilation of the coronary microvasculature is impaired in dilated cardiomyopathy. Circulation 1990 March; 81(3):772-9.
(69) von Schacky C, Baumann K, Angerer P. The effect of n-3 fatty acids on coronary atherosclerosis: results from SCIMO, an angiographic study, background and implications. Lipids 2001; 36 Suppl:S99-102.
(70) Grimble R F, Howell W M, O'Reilly G, Turner S J, Markovic O, Hirrell S, East J M, Calder P C. The ability of fish oil to suppress tumor necrosis factor alpha production by peripheral blood mononuclear cells in healthy men is associated with polymorphisms in genes that influence tumor necrosis factor alpha production. Am J Clin Nutr 2002 August; 76(2):454-9.
(71) Simopoulos A P. Omega-3 fatty acids in inflammation and autoimmune diseases. J Am Coll Nutr 2002 December; 21(6):495-505.
(72) James M J, Gibson R A, Cleland L G. Dietary polyunsaturated fatty acids and inflammatory mediator production. Am J Clin Nutr 2000 January; 71(1 Suppl):343S-8S.
(73) Mozaffarian D, Gottdiener J S, Siscovick D S. Intake of tuna or other broiled or baked fish versus fried fish and cardiac structure, function, and hemodynamics. Am J Cardiol 2006 January 15; 97(2):216-22.
(74) Gissi-Hf I. Effect of rosuvastatin in patients with chronic heart failure (the GISSI-HF trial): a randomised, double-blind, placebo-controlled trial. Lancet 2008 August 29; 372:1231-9.
(75) Fonarow G C. Statins and n-3 fatty acid supplementation in heart failure. Lancet 2008 August 29; 372:1195-6.
(76) Stanley W C, Recchia F A, Lopaschuk G D. Myocardial substrate metabolism in the normal and failing heart. Physiol Rev 2005 July; 85(3):1093-129.
(77) Ashrafian H, Frenneaux M P, Opie L H. Metabolic mechanisms in heart failure. Circulation 2007 July 24; 116(4):434-48.
(78) Lei B, Lionetti V, Young M E, Chandler M P, D′ Agostino C, Kang E, Altarejos M, Matsuo K, Hintze T H, Stanley W C, Recchia F A. Paradoxical downregulation of the glucose oxidation pathway despite enhanced flux in severe heart failure. J Mol Cell Cardiol 2004 April; 36(4):567-76.
(79) Osorio J C, Stanley W C, Linke A, Castellari M, Diep Q N, Panchal A R, Hintze T H, Lopaschuk G D, Recchia F A. Impaired myocardial fatty acid oxidation and reduced protein expression of retinoid X receptor-alpha in pacing-induced heart failure. Circulation 2002 July 30; 106(5):606-12.
(80) Sack M N, Rader T A, Park S, Bastin J, McCune S A, Kelly D P. Fatty acid oxidation enzyme gene expression is downregulated in the failing heart. Circulation 1996 December 1; 94(11):2837-42.
(81) Davila-Roman V G, Vedala G, Herrero P, de las F L, Rogers J G, Kelly D P, Gropler R J. Altered myocardial fatty acid and glucose metabolism in idiopathic dilated cardiomyopathy. J Am Coll Cardiol 2002 July 17; 40(2):271-7.
(82) Huss J M, Kelly D P. Nuclear receptor signaling and cardiac energetics. Circ Res 2004 September 17; 95(6):568-78.
(83) Berger J, Moller D E. The mechanisms of action of PPARs. Annu Rev Med 2002; 53:409-35.
(84) Lehman J J, Kelly D P. Gene regulatory mechanisms governing energy metabolism during cardiac hypertrophic growth. Heart Fail Rev 2002 April; 7(2):175-85.
(85) Harris R A, Huang B, Wu P. Control of pyruvate dehydrogenase kinase gene expression. Adv Enzyme Regul 2001; 41:269-88.
(86) Huang B, Wu P, Bowker-Kinley M M, Harris R A. Regulation of pyruvate dehydrogenase kinase expression by peroxisome proliferator-activated receptor-alpha ligands, glucocorticoids, and insulin. Diabetes 2002 February; 51(2):276-83.
(87) Szczepaniak L S, Victor R G, Orci L, Unger R H. Forgotten but not gone: the rediscovery of Fatty heart, the most common unrecognized disease in america. Circ Res 2007 October 12; 101(8):759-67.
(88) Kliewer S A, Sundseth S S, Jones S A, Brown P J, Wisely G B, Koble C S, Devchand P, Wahli W, Willson T M, Lenhard J M, Lehmann J M. Fatty acids and eicosanoids regulate gene expression through direct interactions with peroxisome proliferator-activated receptors alpha and gamma. Proc Natl Acad Sci USA 1997 April 29; 94(9):4318-23.
(89) Chandler M P, Stanley W C, Morita H, Suzuki G, Roth B A, Blackburn B, Wolff A,
Sabbah H N. Short-term treatment with ranolazine improves mechanical efficiency in dogs with chronic heart failure. Circ Res 2002 August 23; 91(4):278-80.
(90) Imai M, Rastogi S, Sharma N, Chandler M P, Sharov V G, Blackburn B, Belardinelli L, Stanley W C, Sabbah H N. CVT-4325 Inhibits Myocardial Fatty Acid Uptake and Improves Left Ventricular Systolic Function without Increasing Myocardial Oxygen Consumption in Dogs with Chronic Heart Failure. Cardiovasc Drugs Ther 2006 November 27; 21(1):9-15.
(91) Cappola T P, Kass D A, Nelson G S, Berger R D, Rosas G O, Kobeissi Z A, Marban E, Hare J M. Allopurinol improves myocardial efficiency in patients with idiopathic dilated cardiomyopathy. Circulation 2001 November 13; 104(20):2407-11.
(92) Saavedra W F, Paolocci N, St John M E, Skaf M W, Stewart G C, Xie J S, Harrison R W, Zeichner J, Mudrick D, Marban E, Kass D A, Hare J M. Imbalance between xanthine oxidase and nitric oxide synthase signaling pathways underlies mechanoenergetic uncoupling in the failing heart. Circ Res 2002 February 22; 90(3):297-304.
(93) Knaapen P, Germans T, Knuuti J, Paulus W J, Dijkmans P A, Allaart C P, Lammertsma A A, Visser F C. Myocardial energetics and efficiency: current status of the noninvasive approach. Circulation 2007 February 20; 115(7):918-27.
(94) Beanlands R S, Nahmias C, Gordon E, Coates G, deKemp R, Firnau G, Fallen E. The effects of beta(1)-blockade on oxidative metabolism and the metabolic cost of ventricular work in patients with left ventricular dysfunction: A double-blind, placebo-controlled, positron-emission tomography study. Circulation 2000 October 24; 102(17):2070-5.
(95) Sharov V G, Goussev A, Lesch M, Goldstein S, Sabbah H N. Abnormal mitochondrial function in myocardium of dogs with chronic heart failure. J Mol Cell Cardiol 1998 September; 30(9):1757-62.
(96) Sharov V G, Todor A V, Silverman N, Goldstein S, Sabbah H N. Abnormal mitochondrial respiration in failed human myocardium. J Mol Cell Cardiol 2000 December; 32(12):2361-7.
(97) Sabbah H N, Sharov V, Riddle J M, Kono T, Lesch M, Goldstein S. Mitochondrial abnormalities in myocardium of dogs with chronic heart failure. J Mol Cell Cardiol 1992 November; 24(11):1333-47.
(98) Schaper J, Froede R, Hein S, Buck A, Hashizume H, Speiser B, Friedl A, Bleese N. Impairment of the myocardial ultrastructure and changes of the cytoskeleton in dilated cardiomyopathy. Circulation 1991 February; 83(2): 504-14.
(99) Ozcan C, Bienengraeber M, Hodgson D M, Mann D L, Terzic A. Mitochondrial tolerance to stress impaired in failing heart. J Mol Cell Cardiol 2003 September; 35(9):1161-6.
(100) Gong G, Liu J, Liang P, Guo T, Hu Q, Ochiai K, Hou M, Ye Y, Wu X, Mansoor A, From A H, Ugurbil K, Bache R J, Zhang J. Oxidative capacity in failing hearts. Am J Physiol Heart Circ Physiol 2003 August; 285(2):H541-H548.
(101) Sanbe A, Tanonaka K, Kobayasi R, Takeo S. Effects of long-term therapy with ACE inhibitors, captopril, enalapril and trandolapril, on myocardial energy metabolism in rats with heart failure following myocardial infarction. J Mol Cell Cardiol 1995 October; 27(10):2209-22.
(102) Sanbe A, Tanonaka K, Niwano Y, Takeo S. Improvement of cardiac function and myocardial energy metabolism of rats with chronic heart failure by long-term coenzyme Q10 treatment. J Pharmacol Exp Ther 1994 April; 269(1):51-6.
(103) Hoppel C L, Tandler B, Parland W, Turkaly J S, Albers L D. Hamster cardiomyopathy. A defect in oxidative phosphorylation in the cardiac interfibrillar mitochondria. J Biol Chem 1982 February 10; 257(3):1540-8.
(104) Sanbe A, Tanonaka K, Hanaoka Y, Katoh T, Takeo S. Regional energy metabolism of failing hearts following myocardial infarction. J Mol Cell Cardiol 1993 September; 25(9):995-1013.
(105) Javadov S, Purdham D M, Zeidan A, Karmazyn M. NHE-1 inhibition improves cardiac mitochondrial function through regulation of mitochondrial biogenesis during postinfarction remodeling. Am J Physiol Heart Circ Physiol 2006 October; 291(4):H1722-H1730.
(106) Quigley A F, Kapsa R M, Esmore D, Hale G, Byrne E. Mitochondrial respiratory chain activity in idiopathic dilated cardiomyopathy. J Card Fail 2000 March; 6(1):47-55.
(107) Casademont J, Miro O. Electron transport chain defects in heart failure. Heart Fail Rev 2002 April; 7(2):131-9.
(108) Marin-Garcia J, Goldenthal M J, Moe G W. Abnormal cardiac and skeletal muscle mitochondrial function in pacing-induced cardiac failure. Cardiovasc Res 2001 October; 52(1):103-10.
(109) Liu J, Wang C, Murakami Y, Gong G, Ishibashi Y, Prody C, Ochiai K, Bache R J, Godinot C, Zhang J. Mitochondrial ATPase and high-energy phosphates in failing hearts. Am J Physiol Heart Circ Physiol 2001 September; 281(3):H1319-H1326.
(110) Lesnefsky E J, Moghaddas S, Tandler B, Kerner J, Hoppel C L. Mitochondrial dysfunction in cardiac disease: ischemia—reperfusion, aging, and heart failure. J Mol Cell Cardiol 2001 June; 33(6):1065-89.
(111) Stanley W C, Recchia F A, Okere I C. Metabolic therapies for heart disease: fish for prevention and treatment of cardiac failure? Cardiovasc Res 2005 November 1; 68(2):175-7.
(112) Marin-Garcia J, Goldenthal M J, Moe G W. Mitochondrial pathology in cardiac failure. Cardiovasc Res 2001 January; 49(1):17-26.
(113) Scheubel R J, Tostlebe M, Simm A, Rohrbach S, Prondzinsky R, Gellerich F N, Silber R E, Holtz J. Dysfunction of mitochondrial respiratory chain complex I in human failing myocardium is not due to disturbed mitochondrial gene expression. J Am Coll Cardiol 2002 December 18; 40(12):2174-81.
(114) McCutcheon L J, Cory C R, Nowack L, Shen H, Mirsalami M, Lahucky R, Kovac L, O'Grady M, Horne R, O'Brien P J. Respiratory chain defect of myocardial mitochondria in idiopathic dilated cardiomyopathy of Doberman pinscher dogs. Can J Physiol Pharmacol 1992 November; 70(11):1529-33.
(115) Buchwald A, Till H, Unterberg C, Oberschmidt R, Figulla H R, Wiegand V. Alterations of the mitochondrial respiratory chain in human dilated cardiomyopathy. Eur Heart J 1990 June; 11(6):509-16.
(116) Jarreta D, Orus J, Barrientos A, Miro O, Roig E, Heras M, Moraes C T, Cardellach F, Casademont J. Mitochondrial function in heart muscle from patients with idiopathic dilated cardiomyopathy. Cardiovasc Res 2000 March; 45(4):860-5.
(117) Chandler M P, Kerner J, Huang H, Vazquez E, Reszko A, Martini W Z, Hoppel C L, Imai M, Rastogi S, Sabbah H N, Stanley W C. Moderate severity heart failure does not involve a downregulation of myocardial fatty acid oxidation. Am J Physiol Heart Circ Physiol 2004 October; 287(4):H1538-H1543.
(118) Panchal A R, Stanley W C, Kerner J, Sabbah H N. Beta-receptor blockade decreases carnitine palmitoyl transferase I activity in dogs with heart failure. J Card Fail 1998 June; 4(2):121-6.
(119) Schagger H. Respiratory chain supercomplexes. IUBMB Life 2001 September; 52(3-5):119-28.
(120) Dudkina N V, Eubel H, Keegstra W, Boekema E J, Braun H P. Structure of a mitochondrial supercomplex formed by respiratory-chain complexes I and III. Proc Natl Acad Sci USA 2005 March 1; 102(9):3225-9.
(121) Schagger H, Pfeiffer K. Supercomplexes in the respiratory chains of yeast and mammalian mitochondria. EMBO J. 2000 April 17; 19(8):1777-83.
(122) Chicco A J, Sparagna G C, McCune S A, Johnson C A, Murphy R C, Bolden D A, Rees M L, Gardner R T, Moore R L. Linoleate-rich high-fat diet decreases mortality in hypertensive heart failure rats compared with lard and low-fat diets. Hypertension 2008 September; 52(3):549-55.
(123) Sparagna G C, Chicco A J, Murphy R C, Bristow M R, Johnson C A, Rees M L, Maxey M L, McCune S A, Moore R L. Loss of cardiac tetralinoleoyl cardiolipin in human and experimental heart failure. J Lipid Res 2007 July; 48(7):1559-70.
(124) Sparagna G C, Johnson C A, McCune S A, Moore R L, Murphy R C. Quantitation of cardiolipin molecular species in spontaneously hypertensive heart failure rats using electrospray ionization mass spectrometry. J Lipid Res 2005 June; 46(6):1196-204.
(125) Chicco A J, Sparagna G C. Role of cardiolipin alterations in mitochondrial dysfunction and disease. Am J Physiol Cell Physiol 2007 January; 292(1):C33-C44.
(126) Pepe S, Tsuchiya N, Lakatta E G, Hansford R G. PUFA and aging modulate cardiac mitochondrial membrane lipid composition and Ca2+ activation of PDH. Am J Physiol 1999 January; 276(1 Pt 2):H149-H158.
(127) McMillin J B, Bick R J, Benedict C R. Influence of dietary fish oil on mitochondrial function and response to ischemia. Am J Physiol 1992 November; 263(5 Pt 2):H1479-H1485.
(128) Gustafsson A B, Gottlieb R A. Heart mitochondria: gates of life and death. Cardiovasc Res 2008 January 15; 77(2):334-43.
(129) Honda H M, Ping P. Mitochondrial permeability transition in cardiac cell injury and death. Cardiovasc Drugs Ther 2006 December; 20(6):425-32.
(130) Di L F, Bernardi P. Mitochondria and ischemia-reperfusion injury of the heart: fixing a hole. Cardiovasc Res 2006 May 1; 70(2):191-9.
(131) Weiss J N, Korge P, Honda H M, Ping P. Role of the mitochondrial permeability transition in myocardial disease. Circ Res 2003 August 22; 93(4):292-301.
(132) Halestrap A P, Pasdois P. The role of the mitochondrial permeability transition pore in heart disease. Biochim Biophys Acta 2009 Jan. 8.
(133) Baines C P, Kaiser R A, Sheiko T, Craigen W J, Molkentin J D. Voltage-dependent anion channels are dispensable for mitochondrial-dependent cell death. Nat Cell Biol 2007 May; 9(5):550-5.
(134) Leung A W, Halestrap A P. Recent progress in elucidating the molecular mechanism of the mitochondrial permeability transition pore. Biochim Biophys Acta 2008 July; 1777(7-8):946-52.
(135) Marcil M, Ascah A, Matas J, Belanger S, Deschepper C F, Burelle Y. Compensated volume overload increases the vulnerability of heart mitochondria without affecting their functions in the absence of stress. J Mol Cell Cardiol 2006 December; 41(6):998-1009.
(136) Matas J, Tien Sing Y N, Bourcier-Lucas C, Ascah A, Marcil M, Deschepper C F, Burelle Y. Increased expression and intramitochondrial translocation of cyclophilin-D associates with increased vulnerability of the permeability transition pore to stress-induced opening during compensated ventricular hypertrophy. J Mol Cell Cardiol 2008 Nov. 6.
(137) Nakayama H, Chen X, Baines C P, Klevitsky R, Zhang X, Zhang H, Jaleel N, Chua B H, Hewett T E, Robbins J, Houser S R, Molkentin J D. Ca2+- and mitochondrial-dependent cardiomyocyte necrosis as a primary mediator of heart failure. J Clin Invest 2007 September; 117(9):2431-44.
(138) Sharov V G, Sabbah H N, Shimoyama H, Goussev A V, Lesch M, Goldstein S. Evidence of cardiocyte apoptosis in myocardium of dogs with chronic heart failure. Am J Pathol 1996 January; 148(1):141-9.
(139) Todor A, Sharov V G, Tanhehco E J, Silverman N, Bernabei A, Sabbah H N. Hypoxia-induced cleavage of caspase-3 and DFF45/ICAD in human failed cardiomyocytes. Am J Physiol Heart Circ Physiol 2002 September; 283(3):H990-H995.
(140) Kang P M, Yue P, Liu Z, Tarnayski O, Bodyak N, Izumo S. Alterations in apoptosis regulatory factors during hypertrophy and heart failure. Am J Physiol Heart Circ Physiol 2004 July; 287(1):H72-H80.
(141) Barouch L A, Gao D, Chen L, Miller K L, Xu W, Phan A C, Kittleson M M, Minhas K M, Berkowitz D E, Wei C, Hare J M. Cardiac myocyte apoptosis is associated with increased DNA damage and decreased survival in murine models of obesity. Circ Res 2006 January 6; 98(1):119-24.
(142) Foo R S, Mani K, Kitsis R N. Death begets failure in the heart. J Clin Invest 2005 March; 115(3):565-71.
(143) van E, V, Bertrand A T, Hofstra L, Crijns H J, Doevendans P A, De Windt L J. Myocyte apoptosis in heart failure. Cardiovasc Res 2005 July 1; 67(1):21-9.
(144) Olivetti G, Abbi R, Quaini F, Kajstura J, Cheng W, Nitahara J A, Quaini E, Di L C, Beltrami C A, Krajewski S, Reed J C, Anversa P. Apoptosis in the failing human heart. N Engl J Med 1997 April 17; 336(16):1131-41.
(145) Saraste A, Pulkki K, Kallajoki M, Heikkila P, Laine P, Mattila S, Nieminen M S, Parvinen M, Voipio-Pulkki L M. Cardiomyocyte apoptosis and progression of heart failure to transplantation. Eur J Clin Invest 1999 May; 29(5):380-6.
(146) Guerra S, Leri A, Wang X, Finato N, Di L C, Beltrami C A, Kajstura J, Anversa P. Myocyte death in the failing human heart is gender dependent. Circ Res 1999 October 29; 85(9):856-66.
(147) Sabbah H N, Sharov V G, Gupta R C, Todor A, Singh V, Goldstein S. Chronic therapy with metoprolol attenuates cardiomyocyte apoptosis in dogs with heart failure. J Am Coll Cardiol 2000 November 1; 36(5):1698-705.
(148) Goussev A, Sharov V G, Shimoyama H, Tanimura M, Lesch M, Goldstein S, Sabbah H N. Effects of ACE inhibition on cardiomyocyte apoptosis in dogs with heart failure. Am J Physiol 1998 August; 275(2 Pt 2):H626-H631.
(149) Hunter J J, Chien K R. Signaling pathways for cardiac hypertrophy and failure. N Engl J Med 1999 October 21; 341(17):1276-83.
(150) Okere I C, Chandler M P, McElfresh T A, Rennison J H, Sharov V, Sabbah H N, Tserng K Y, Hoit B D, Ernsberger P, Young M E, Stanley W C. Differential effects of saturated and unsaturated fatty acid diets on cardiomyocyte apoptosis, adipose distribution, and serum leptin. Am J Physiol Heart Circ Physiol 2006 July; 291(1):H38-H44.
(151) Sparagna G C, Hickson-Bick D L, Buja L M, McMillin J B. A metabolic role for mitochondria in palmitate-induced cardiac myocyte apoptosis. Am J Physiol Heart Circ Physiol 2000 November; 279(5):H2124-H2132.
(152) Miller T A, LeBrasseur N K, Cote G M, Trucillo M P, Pimentel D R, Ido Y, Ruderman N B, Sawyer D B. Oleate prevents palmitate-induced cytotoxic stress in cardiac myocytes. Biochem Biophys Res Commun 2005 October 14; 336(1):309-15.
(153) Listenberger L L, Ory D S, Schaffer J E. Palmitate-induced apoptosis can occur through a ceramide-independent pathway. J Biol Chem 2001 May 4; 276(18):14890-5.
(154) Kong J Y, Rabkin S W. Palmitate-induced apoptosis in cardiomyocytes is mediated through alterations in mitochondria: prevention by cyclosporin A. Biochim Biophys Acta 2000 May 6; 1485(1):45-55.
(155) Hickson-Bick D L, Sparagna G C, Buja L M, McMillin J B. Palmitate-induced apoptosis in neonatal cardiomyocytes is not dependent on the generation of ROS. Am J Physiol Heart Circ Physiol 2002 February; 282(2):H656-H664.
(156) Kong J Y, Rabkin S W. Palmitate-induced cardiac apoptosis is mediated through CPT-1 but not influenced by glucose and insulin. Am J Physiol Heart Circ Physiol 2002 February; 282(2):H717-H725.
(157) Hickson-Bick D L, Buja M L, McMillin J B. Palmitate-mediated alterations in the fatty acid metabolism of rat neonatal cardiac myocytes. J Mol Cell Cardiol 2000 March; 32(3):511-9.
(158) Leroy C, Tricot S, Lacour B, Grynberg A. Protective effect of eicosapentaenoic acid on palmitate-induced apoptosis in neonatal cardiomyocytes. Biochim Biophys Acta 2008 Aug. 8.
(159) El Assaad W, Buteau J, Peyot M L, Nolan C, Roduit R, Hardy S, Joly E, Dbaibo G, Rosenberg L, Prentki M. Saturated fatty acids synergize with elevated glucose to cause pancreatic beta-cell death. Endocrinology 2003 September; 144(9):4154-63.
(160) Schlame M, Ren M, Xu Y, Greenberg M L, Haller I. Molecular symmetry in mitochondrial cardiolipins. Chem Phys Lipids 2005 December; 138(1-2):38-49.
(161) Schlame M, Ren M. Barth syndrome, a human disorder of cardiolipin metabolism. FEBS Lett 2006 October 9; 580(23):5450-5.
(162) Schlame M, Towbin J A, Heerdt P M, Jehle R, DiMauro S, Blanck T J. Deficiency of tetralinoleoyl-cardiolipin in Barth syndrome. Ann Neurol 2002 May; 51(5):634-7.
(163) Schlame M, Kelley R I, Feigenbaum A, Towbin J A, Heerdt P M, Schieble T, Wanders R J, DiMauro S, Blanck T J. Phospholipid abnormalities in children with Barth syndrome. J Am Coll Cardiol 2003 December 3; 42(11):1994-9.
(164) Lee H J, Mayette J, Rapoport S I, Bazinet R P. Selective remodeling of cardiolipin fatty acids in the aged rat heart. Lipids Health Dis 2006; 5:2.
(165) Yamaoka S, Urade R, Kito M. Cardiolipin molecular species in rat heart mitochondria are sensitive to essential fatty acid-deficient dietary lipids. J Nutr 1990 May; 120(5):415-21.
(166) Yamaoka-Koseki S, Urade R, Kito M. Cardiolipins from rats fed different dietary lipids affect bovine heart cytochrome c oxidase activity. J Nutr 1991 July; 121(7):956-8.
(167) Paradies G, Ruggiero F M, Petrosillo G, Quagliariello E. Age-dependent decline in the cytochrome c oxidase activity in rat heart mitochondria: role of cardiolipin. FEBS Lett 1997 April 7; 406(1-2):136-8.
(168) Pepe S. Effect of dietary polyunsaturated fatty acids on age-related changes in cardiac mitochondrial membranes. Exp Gerontol 2005 May; 40(5):369-76.
(169) Moghaddas S, Stoll M S, Minkler P E, Salomon R G, Hoppel C L, Lesnefsky E J. Preservation of cardiolipin content during aging in rat heart interfibrillar mitochondria. J Gerontol A Biol Sci Med Sci 2002 January; 57(1):B22-B28.
(170) Tuominen E K, Wallace C J, Kinnunen P K. Phospholipid-cytochrome c interaction: evidence for the extended lipid anchorage. J Biol Chem 2002 March 15; 277(11):8822-6.
(171) Ott M, Robertson J D, Gogvadze V, Zhivotovsky B, Orrenius S. Cytochrome c release from mitochondria proceeds by a two-step process. Proc Natl Acad Sci USA 2002 February 5; 99(3):1259-63.
(172) Campos H, Baylin A, Willett W C. Alpha-linolenic acid and risk of nonfatal acute myocardial infarction. Circulation 2008 July 22; 118(4):339-45.
(173) Djousse L, Arnett D K, Can J J, Eckfeldt J H, Hopkins P N, Province M A, Ellison R C. Dietary linolenic acid is inversely associated with calcified atherosclerotic plaque in the coronary arteries: the National Heart, Lung, and Blood Institute Family Heart Study. Circulation 2005 June 7; 111(22):2921-6.
(174) De L M, Renaud S, Mamelle N, Salen P, Martin J L, Monjaud I, Guidollet J, Touboul P, Delaye J. Mediterranean alpha-linolenic acid-rich diet in secondary prevention of coronary heart disease. Lancet 1994 June 11; 343(8911):1454-9.
(175) Hu F B, Stampfer M J, Manson J E, Rimm E B, Wolk A, Colditz G A, Hennekens C H, Willett W C. Dietary intake of alpha-linolenic acid and risk of fatal ischemic heart disease among women. Am J Clin Nutr 1999 May; 69(5):890-7.
(176) Baylin A, Kabagambe E K, Ascherio A, Spiegelman D, Campos H. Adipose tissue alpha-linolenic acid and nonfatal acute myocardial infarction in Costa Rica. Circulation 2003 April 1; 107(12):1586-91.
(177) Guivernau M, Meza N, Barja P, Roman O. Clinical and experimental study on the long-term effect of dietary gamma-linolenic acid on plasma lipids, platelet aggregation, thromboxane formation, and prostacyclin production. Prostaglandins Leukot Essent Fatty Acids 1994 November; 51(5):311-6.
(178) Djousse L, Hunt S C, Arnett D K, Province M A, Eckfeldt J H, Ellison R C. Dietary linolenic acid is inversely associated with plasma triacylglycerol: the National Heart, Lung, and Blood Institute Family Heart Study. Am J Clin Nutr 2003 December; 78(6):1098-102.
(179) Caughey G E, Mantzioris E, Gibson R A, Cleland L G, James M J. The effect on human tumor necrosis factor alpha and interleukin 1 beta production of diets enriched in n-3 fatty acids from vegetable oil or fish oil. Am J Clin Nutr 1996 January; 63(1):116-22.
(180) Muders F, Elsner D. Animal models of chronic heart failure. Pharmacol Res 2000 June; 41(6):605-12.
(181) Recchia F A, Lionetti V. Animal models of dilated cardiomyopathy for translational research. Vet Res Commun 2007 August; 31 Suppl 1:35-41.
(182) Arnolda L F, Llewellyn-Smith U, Minson J B. Animal models of heart failure. Aust N Z J Med 1999 June; 29(3):403-9.
(183) Liu Y H, Yang X P, Nass O, Sabbah H N, Peterson E, Carretero O A. Chronic heart failure induced by coronary artery ligation in Lewis inbred rats. Am J Physiol 1997 February; 272(2 Pt 2):H722-H727.
(184) Sabbah H N, Stein P D, Kono T, Gheorghiade M, Levine T B, Jafri S, Hawkins E T, Goldstein S. A canine model of chronic heart failure produced by multiple sequential coronary microembolizations. Am J Physiol 1991 April; 260(4 Pt 2):H1379-H1384.
(185) Power J M, Tonkin A M. Large animal models of heart failure. Aust N Z J Med 1999 June; 29(3):395-402.
(186) Lennie T A, Chung M L, Habash D L, Moser D K. Dietary fat intake and proinflammatory cytokine levels in patients with heart failure. J Card Fail 2005 October; 11(8):613-8.
(187) Siena S, Lara-Villoslada F, Comalada M, Olivares M, Xaus J. Dietary fish oil n-3 fatty acids increase regulatory cytokine production and exert anti-inflammatory effects in two murine models of inflammation. Lipids 2006 December; 41(12):1115-25.
(188) Matsuyama W, Mitsuyama H, Watanabe M, Oonakahara K, Higashimoto I, Osame M, Arimura K. Effects of omega-3 polyunsaturated fatty acids on inflammatory markers in COPD. Chest 2005 December; 128(6):3817-27.
(189) Lee K W, Blann A D, Lip G Y. Effects of omega-3 polyunsaturated fatty acids on plasma indices of thrombogenesis and inflammation in patients post-myocardial infarction. Thromb Res 2006; 118(3):305-12.
(190) De L M. Essential polyunsaturated fatty acids, inflammation, atherosclerosis and cardiovascular diseases. Subcell Biochem 2007; 42:283-97.
(191) Calder P C. Polyunsaturated fatty acids and inflammation. Prostaglandins Leukot Essent Fatty Acids 2006 September; 75(3):197-202.
(192) Ferrucci L, Cherubini A, Bandinelli S, Bartali B, Corsi A, Lauretani F, Martin A, ndres-Lacueva C, Senin U, Guralnik J M. Relationship of plasma polyunsaturated fatty acids to circulating inflammatory markers. J Clin Endocrinol Metab 2006 February; 91(2):439-46.
(193) Mann D L. Stress-activated cytokines and the heart: from adaptation to maladaptation. Annu Rev Physiol 2003; 65:81-101.
(194) Bozkurt B, Torre-Amione G, Warren M S, Whitmore J, Soran O Z, Feldman A M, Mann D L. Results of targeted anti-tumor necrosis factor therapy with etanercept (ENBREL) in patients with advanced heart failure. Circulation 2001 February 27; 103(8):1044-7.
(195) Gullestad L, Kjekshus J, Damas J K, Ueland T, Yndestad A, Aukrust P. Agents targeting inflammation in heart failure. Expert Opin Investig Drugs 2005 May; 14(5):557-66.
(196) Aukrust P, Gullestad L, Ueland T, Damas J K, Yndestad A. Inflammatory and anti-inflammatory cytokines in chronic heart failure: potential therapeutic implications. Ann Med 2005; 37(2):74-85.
(197) Mann D L, McMurray J J, Packer M, Swedberg K, Borer J S, Colucci W S, Djian J, Drexler H, Feldman A, Kober L, Krum H, Liu P, Nieminen M, Tavazzi L, van Veldhuisen D J, Waldenstrom A, Warren M, Westheim A, Zannad F, Fleming T. Targeted anticytokine therapy in patients with chronic heart failure: results of the Randomized Etanercept Worldwide Evaluation (RENEWAL). Circulation 2004 April 6; 109(13):1594-602.
(198) Zaca V, Rastogi S, Imai M, Wang M, Sharov V G, Jiang A, Goldstein S, Sabbah H N. Chronic monotherapy with rosuvastatin prevents progressive left ventricular dysfunction and remodeling in dogs with heart failure. J Am Coll Cardiol 2007 August 7; 50(6):551-7.
(199) Skudicky D, Bergemann A, Sliwa K, Candy G, Sareli P. Beneficial effects of pentoxifylline in patients with idiopathic dilated cardiomyopathy treated with angiotensin-converting enzyme inhibitors and carvedilol: results of a randomized study. Circulation 2001 February 27; 103(8):1083-8.
(200) Sliwa K, Woodiwiss A, Candy G, Badenhorst D, Libhaber C, Norton G, Skudicky D, Sareli P. Effects of pentoxifylline on cytokine profiles and left ventricular performance in patients with decompensated congestive heart failure secondary to idiopathic dilated cardiomyopathy. Am J Cardiol 2002 November 15; 90(10):1118-22.
(201) Bahrmann P, Hengst U M, Richartz B M, Figulla H R. Pentoxifylline in ischemic, hypertensive and idiopathic-dilated cardiomyopathy: effects on left-ventricular function, inflammatory cytokines and symptoms. Eur J Heart Fail 2004 March 1; 6(2):195-201.
(202) Sliwa K, Skudicky D, Candy G, Wisenbaugh T, Sareli P. Randomised investigation of effects of pentoxifylline on left-ventricular performance in idiopathic dilated cardiomyopathy. Lancet 1998 April 11; 351(9109):1091-3.
(203) Sliwa K, Woodiwiss A, Kone V N, Candy G, Badenhorst D, Norton G, Zambakides C, Peters F, Essop R. Therapy of ischemic cardiomyopathy with the immunomodulating agent pentoxifylline: results of a randomized study. Circulation 2004 February 17; 109(6):750-5.
(204) Guggilam A, Hague M, Kerut E K, McIlwain E, Lucchesi P, Seghal I, Francis J. TNF-alpha blockade decreases oxidative stress in the paraventricular nucleus and attenuates sympathoexcitation in heart failure rats. Am J Physiol Heart Circ Physiol 2007 July; 293(1):H599-H609.
(205) Shibata R, Sato K, Pimentel D R, Takemura Y, Kihara S, Ohashi K, Funahashi T, Ouchi N, Walsh K. Adiponectin protects against myocardial ischemia-reperfusion injury through. Nat Med 2005 October; 11(10):1096-103.
(206) Shibata R, Ouchi N, Ito M, Kihara S, Shiojima I, Pimentel D R, Kumada M, Sato K, Schiekofer S, Ohashi K, Funahashi T, Colucci W S, Walsh K. Adiponectin-mediated modulation of hypertrophic signals in the heart. Nat Med 2004 December; 10(12): 1384-9.
(207) De Sousa E, Lechene P, Fortin D, N'Guessan B, Belmadani S, Bigard X, Veksler V, Ventura-Clapier R. Cardiac and skeletal muscle energy metabolism in heart failure: beneficial effects of voluntary activity. Cardiovasc Res 2002 November; 56(2):260-8.
(208) Garnier A, Fortin D, Delomenie C, Momken I, Veksler V, Ventura-Clapier R. Depressed mitochondrial transcription factors and oxidative capacity in rat failing cardiac and skeletal muscles. J Physiol 2003 September 1; 551(Pt 2):491-501.
(209) Momken I, Kahapip J, Bahi L, Badoual T, Hittinger L, Ventura-Clapier R, Veksler V. Does angiotensin-converting enzyme inhibition improve the energetic status of cardiac and skeletal muscles in heart failure induced by aortic stenosis in rats? J Mol Cell Cardiol 2003 April; 35(4):399-407.
(210) Ciobotaru V, Heimburger M, Louedec L, Heymes C, Ventura-Clapier R, Bedossa P, Escoubet B, Michel J B, Mercadier J J, Logeart D. Effect of long-term heart rate reduction by If current inhibition on pressure overload-induced heart failure in rats. J Pharmacol Exp Ther 2008 January; 324(1):43-9.
(211) Joubert F, Wilding J R, Fortin D, Domergue-Dupont V, Novotova M, Ventura-Clapier R, Veksler V. Local energetic regulation of sarcoplasmic and myosin ATPase is differently impaired in rats with heart failure. J Physiol 2008 November 1; 586(Pt 21):5181-92.
(212) Duda M K, O'shea KM, Lei B, Barrows B R, Azimzadeh A M, McElfresh T E, Hoit B D, Kop W J, Stanley W C. Dietary supplementation with omega-3 PUFA increases adiponectin and attenuates ventricular remodeling and dysfunction with pressure overload. Cardiovasc Res 2007 July 20; 76:303-10.
(213) Duda M K, O'shea KM, Lei B, Barrows B R, Azimzadeh A M, McElfresh T E, Hoit B D, Kop W J, Stanley W C. Low-carbohydrate/high-fat diet attenuates pressure overload-induced ventricular remodeling and dysfunction. J Card Fail 2008 May; 14(4):327-35.
(214) Okere I C, Young M E, McElfresh T A, Chess D J, Sharov V G, Sabbah H N, Hoit B D, Ernsberger P, Chandler M P, Stanley W C. Low carbohydrate/high-fat diet attenuates cardiac hypertrophy, remodeling, and altered gene expression in hypertension. Hypertension 2006 December; 48(6):1116-23.
(215) King K L, Young M E, Kerner J, Huang H, O'shea K M, Alexson S E, Hoppel C L, Stanley W C. Diabetes and activation of peroxisome proliferator activated receptor alpha increase mitochondrial thioesterase I protein expression and activity in the heart. J Lipid Res 2007 Apr. 16.
(216) Schlame M, Rua D, Greenberg M L. The biosynthesis and functional role of cardiolipin. Prog Lipid Res 2000 May; 39(3):257-88.
(217) Gottlieb R A. Mitochondrial signaling in apoptosis: mitochondrial daggers to the breaking heart. Basic Res Cardiol 2003 July; 98(4):242-9.
(218) Kristian T, Gertsch J, Bates T E, Siesjo B K. Characteristics of the calcium-triggered mitochondrial permeability transition in nonsynaptic brain mitochondria: effect of cyclosporin A and ubiquinone O. J Neurochem 2000 May; 74(5):1999-2009.
(219) Bopassa J C, Vandroux D, Ovize M, Ferrera R. Controlled reperfusion after hypothermic heart preservation inhibits mitochondrial permeability transition-pore opening and enhances functional recovery. Am J Physiol Heart Circ Physiol 2006 November; 291(5):H2265-H2271.
(220) Marcil M, Bourduas K, Ascah A, Burelle Y. Exercise training induces respiratory substrate-specific decrease in Ca2+-induced permeability transition pore opening in heart mitochondria. Am J Physiol Heart Circ Physiol 2006 April; 290(4):H1549-H1557.
(221) Lange C, Nett J H, Trumpower B L, Hunte C. Specific roles of protein-phospholipid interactions in the yeast cytochrome bc1 complex structure. EMBO J. 2001 December 3; 20(23):6591-600.
(222) C. P. Baines, R. A. Kaiser, N. H. Purcell, N. S. Blair, H. Osinska, M. A. Hambleton, E. W. Brunskill, M. R. Sayen, R. A. Gottlieb, G. W. Dorn, J. Robbins, J. D. Molkentin, Loss of cyclophilin D reveals a critical role for mitochondrial permeability transition in cell death, Nature 434 (2005) pp. 658-662.
(223) E. Basso, V. Petronilli, M. A. Forte, P. Bernardi, Phosphate is essential for inhibition of the mitochondrial permeability transition pore by cyclosporin A and by cyclophilin D ablation, J. Biol. Chem. 283 (2008) pp. 26307-26311.
(224) S. Grimsgaard, K. H. Bonaa, J. B. Hansen, A. Nordoy, Highly purified eicosapentaenoic acid and docosahexaenoic acid in humans have similar triacylglycerol-lowering effects but divergent effects on serum fatty acids, Am J. Clin. Nutr. 66 (1997) pp. 649-659.
(225) T. A. Jacobson, Role of n-3 fatty acids in the treatment of hypertriglyceridemia and cardiovascular disease, Am J. Clin. Nutr. 87 (2008) pp. 1981S-1990S.
(226) T. Kristian, J. Gertsch, T. E. Bates, B. K. Siesjo, Characteristics of the calcium-triggered mitochondrial permeability transition in nonsynaptic brain mitochondria: effect of cyclosporin A and ubiquinone O, J Neurochem. 74 (2000) pp. 1999-2009.
(227) T. A. Mori and R. J. Woodman, The independent effects of eicosapentaenoic acid and docosahexaenoic acid on cardiovascular risk factors in humans, Curr. Opin. Clin. Nutr. Metab Care 9 (2006) pp. 95-104.
(228) K. M. O'shea, R. J. Khairallah, G. C. Sparagna, W. Xu, P. A. Hecker, I. Robillard-Frayne, R. C. des, T. Kristian, R. C. Murphy, G. Fiskum, W. C. Stanley, Dietary omega-3 fatty acids alter cardiac mitochondrial phospholipid composition and delay Ca2+-induced permeability transition, J. Mol. Cell. Cardiol. 47 (2009) pp. 819-827.
(229) K. B. Shah, M. K. Duda, K. M. O'shea, G. C. Sparagna, D. J. Chess, R. J. Khairallah, I. Robillard-Frayne, W. Xu, R. C. Murphy, R. C. des, W. C. Stanley, The cardioprotective effects of fish oil during pressure overload are blocked by high fat intake: role of cardiac phospholipid remodeling, Hypertension 54 (2009) pp. 605-611.
(230) G. C. Sparagna, C. A. Johnson, S. A. McCune, R. L. Moore, R. C. Murphy, Quantitation of cardiolipin molecular species in spontaneously hypertensive heart failure rats using electrospray ionization mass spectrometry, J Lipid Res 46 (2005) pp. 1196-1204.
(231) G. C. Sparagna and E. J. Lesnefsky, Cardiolipin remodeling in the heart, J Cardiovasc Pharmacol. 53 (2009) pp. 290-301.

1. A method of treating heart failure in a subject, comprising administering a pharmaceutical composition consisting essentially of a therapeutically effective amount of docosahexaenoic acid (DHA) to a subject in need of treatment.

2. The method of claim 1, wherein said treating has one or more effects selected from the group consisting of preserving cardiac mitochondrial function, maintaining cardiac respiratory supercomplexes, increasing cardiac respiratory supercomplexes, maintaining cardiac oxidative phosphorylation, increasing cardiac oxidative phosphorylation, preventing cardiac MPTP opening, maintaining cardiac levels of DHA, increasing cardiac levels of DHA, maintaining levels of EPA, increasing levels of EPA, maintaining levels of cardiac mitochondrial cardiolipin, increasing levels of cardiac mitochondrial cardiolipin, inhibiting cardiomyocyte death by apoptosis and inhibiting cardiomyocyte death by necrosis.

3. The method of claim 1, wherein said treating contributes to one or more clinical end points selected from the group consisting of reduced left ventricular volume, improved cardiac contractile function, reduced cardiac-related hospitalization, reduced cardiac-medical complications, improved quality of life and reduced mortality.

4. The method of claim 1, wherein the therapeutically effective amount of DHA is between about 0.5 g and about 5 g, and the pharmaceutical composition is administered to the subject once or twice daily.

5. The method of claim 1, wherein the pharmaceutical composition is administered to the subject as an oral formulation.

6. The method of claim 4, wherein the pharmaceutical composition is administered to the subject as an oral formulation.

7. A method of increasing cardiac mitochondrial DHA, EPA or cardiolipin in a subject, comprising administering a pharmaceutical composition consisting essentially of a therapeutically effective amount of DHA to a subject in need thereof.

8. The method of claim 7, wherein the therapeutically effective amount of DHA is between about 0.5 g and about 5 g, and the pharmaceutical composition is administered to the subject once or twice daily.

9. The method of claim 7, wherein the pharmaceutical composition is administered to the subject as an oral formulation.

10. A method of suppressing cardiac MPTP opening in a subject, comprising administering a pharmaceutical composition consisting essentially of therapeutically effective amount of DHA to a subject in need thereof.

11. The method of claim 10, wherein the therapeutically effective amount of DHA is between about 0.5 g and about 5 g, and the pharmaceutical composition is administered to the subject once or twice daily.

12. The method of claim 10, wherein the pharmaceutical composition is administered to the subject as an oral formulation.

13-15. (canceled)

16. The method of claim 2, wherein the therapeutically effective amount of DHA is between about 0.5 g and about 5 g, and the pharmaceutical composition is administered to the subject once or twice daily.

17. The method of claim 3, wherein the therapeutically effective amount of DHA is between about 0.5 g and about 5 g, and the pharmaceutical composition is administered to the subject once or twice daily.

18. The method of claim 2, wherein the pharmaceutical composition is administered to the subject as an oral formulation.

19. The method of claim 3, wherein the pharmaceutical composition is administered to the subject as an oral formulation.

20. The method of claim 8, wherein the pharmaceutical composition is administered to the subject as an oral formulation.

21. The method of claim 11, wherein the pharmaceutical composition is administered to the subject as an oral formulation.
